(19) United States
(12) Patent Application Publication
(10) Pub. No.: US 2014/0072099 A1
(43) Pub. Date: Mar. 13, 2014

(54) X-RAY CT APPARATUS, X-RAY CT SYSTEM

(71) Applicants: Toshiba Medical Systems Corporation, Otawara-shi (JP); Kabushiki Kaisha Toshiba, Minato-ku (JP)

(72) Inventors: Go Mukumoto, Utsunomiya-shi (JP); Manabu Hiraoka, Nasushiobara-shi (JP)

(73) Assignees: Kabushiki Kaisha Toshiba, Minato-ku, Tokyo (JP); Toshiba Medical Systems Corporation, Otawara-shi (JP)

(21) Appl. No.: 14/111,965

(22) PCT Filed: Jan. 23, 2013
(86) PCT No.: PCT/JP2013/051356

§ 371 Date: Oct. 15, 2013

(30) Foreign Application Priority Data
Jan. 27, 2012 (JP) 2012-015078
Jan. 27, 2012 (JP) 2012-015084

(51) Int. Cl. A61B 6/03

(52) U.S. Cl. CPC A61B 6/03
USPC 378/19; 378/4

(57) ABSTRACT

A technique is provided that makes it possible to easily recognize the pre-defined image on the image obtained in the present. An X-ray CT apparatus creates first volume data and second volume data based on the results of scanning a subject with X-rays at different timings. The X-ray CT apparatus comprises: a setter, a storage and a display controller. The setter is configured to set a specified setup image in regard to an image based on the first volume data. The storage is configured to store the setup image and a setup position thereof. The display controller is configured to cause a display to display an image based on the second volume data, as well as causing the setup image to be displayed in the position corresponding to the setup position in the image based on the second volume data.

### FIELD OF THE INVENTION

The embodiments the present invention relates to an X-ray CT apparatus and X-ray CT system.

### BACKGROUND ART

An X-ray CT (Computed Tomography) apparatus is an apparatus that scans a subject using X-rays to acquire data, and then processes the acquired data using a computer in order to generate an internal image of the subject.

Specifically, an X-ray CT apparatus exposes X-rays onto the subject from different angles multiple times in a circular path around the subject. The X-ray CT apparatus detects the X-rays passing through the subject with an X-ray detector, and acquires multiple detection data. The acquired detection data is A/D converted by a data acquisition system before being transmitted to a console device. The console device pre-processes, or the like, the detection data to form projection data. Next, the console device implements reconstruction processing based on the projection data to form tomographic image data, or volume data based on multiple sets of tomographic image data. The volume data is a data set that expresses the three-dimensional CT value distribution corresponding to the three-dimensional area of the subject.

The X-ray CT apparatus may display an MPR (Multi Planar Reconstruction) as a result of rendering the aforementioned volume data in an arbitrary direction. Hereinafter, the cross-sectional image displayed as MPR as a result of rendering the volume data is referred to, in some cases, as a “MPR image”. MPR images may include, for example, axial images, which depict an orthogonal cross-section with respect to a body axis, sagittal images, which depict a vertical cross-section along the body axis of the subject, and coronal images, which depict a horizontal cross-section along the body axis of the subject. Furthermore, MPR images also include images taken at an arbitrary cross section within the volume data (oblique images). The created multiple MPR images may be displayed simultaneously on a display, or the like.

CT fluoroscopy (CTF: Computed Tomography Fluoroscopy) is an imaging method implemented using an X-ray CT apparatus. CT fluoroscopy is an imaging method whereby a subject is irradiated continuously with X-rays to obtain a real time image of the area of interest within the subject. In CT fluoroscopy, shortening the acquisition rate of the detection data and the time required for reconstruction processing allows the image to be created in real time. CT fluoroscopy is used, for example, to confirm the positional relationship between the tip of a puncture needle used in biopsy and a site from which a specimen is being collected, to confirm the position of a tube used when the drainage method is implemented, and the like. The drainage method is a method of removing fluid pooled in a body cavity using a tube, or the like.

When implementing a biopsy of a subject while referring to MPR images based on volume data obtained using CT fluoroscopy, for example, scanning and puncturing are sometimes implemented alternately. Specifically, an MPR image is first obtained of the subject using CT fluoroscopy. The doctor and the like, implement puncturing while referring to the MPR image. At this point, in order to confirm the positional relationship between the tip of a puncture needle and a site from which a specimen is being collected, further CT fluoroscopy is implemented after the puncture has been carried out to a certain extent. The doctor and the like then proceed with puncturing while referring to the MPR image obtained during the repeated CT fluoroscopy. Repeating this process until the biopsy is completed facilitates the accurate implementation of the biopsy.

Additionally, when implementing a biopsy using CT fluoroscopy, sometimes a puncture plan is created in advance. The puncture plan includes information relating to a preset puncture needle insertion route in regard to the subject (hereinafter, referred to in some cases as the “planned route”). The puncture plan is defined by, for example, using input instructions via a mouse, and the like to draw the planned route onto a CT image pre-acquired before CT fluoroscopy is carried out. The doctor and the like implements puncturing of the subject while referring to the CT image depicting the planned route, as well as the MPR image based on the volume data obtained each time X-ray scanning is performed.

### PRIOR ART DOCUMENT

### Patent Document

[Patent Document 1] Japanese Unexamined Patent Application Publication No. 2002-112998

### SUMMARY OF THE INVENTION

### Problem to be Solved by the Invention

The image (for example, planned route) defined within the pre-acquired CT image, however, is not displayed on the image based on volume data obtained each time X-ray scanning is performed.

The embodiments of the present invention are intended to solve the aforementioned problem by providing a technique that makes it possible to easily recognize the pre-defined image on the image obtained in the present.

### Means of Solving the Problem

An X-ray CT apparatus of this embodiment creates first volume data and second volume data based on the results of scanning a subject with X-rays at different timings. The X-ray CT apparatus comprises: a setter, a storage and a display controller. The setter is configured to set a specified setup image in regard to an image based on the first volume data. The storage is configured to store the setup image and a setup position thereof. The display controller is configured to cause a display to display an image based on the second volume data, as well as causing the setup image to be displayed in the position corresponding to the setup position in the image based on the second volume data.

An X-ray CT system of another embodiment comprises an X-ray CT apparatus configured to produce volume data based on the results of scanning a subject with X-rays. The X-ray CT system comprises: a setter, a storage, and a display controller. The setter is configured to set a specified setup image in regard to an image based on a pre-created first volume data. The storage is configured to store the setup image and a setup position thereof. The display controller is configured to cause the display to display an image based on a newly created second volume data, as well as causing the display of the setup image in the position corresponding to the setup position in the image based on the second volume data.

### BRIEF DESCRIPTION OF THE DRAWINGS

FIG. 1 is a block diagram illustrating an X-ray CT apparatus in a first embodiment.

FIG. 2A is a diagram providing further illustration of a setter in the first embodiment.

FIG. 2B is a diagram providing further illustration of the setter in the first embodiment.

FIG. 3 is a flow chart outlining the operation of the X-ray CT apparatus in the first embodiment.

FIG. 4A is a diagram providing further illustration of a setter in a second embodiment.

FIG. 4B is a diagram providing further illustration of the setter in the second embodiment.

FIG. 5 is a flow chart outlining the operation of an X-ray CT apparatus in the second embodiment.

FIG. 6 is a block diagram illustrating an X-ray CT apparatus in a third embodiment.

FIG. 7A is a diagram providing further illustration of a first setter in the third embodiment.

FIG. 7B is a diagram providing further illustration of the first setter in the third embodiment.

FIG. 7C is a diagram providing further illustration of a second setter in the third embodiment.

FIG. 7 D is a diagram providing further illustration of the second setter in the third embodiment.

FIG. 8A is a diagram providing further illustration of the second setter in the third embodiment.

FIG. 8B is a diagram providing further illustration of the second setter in the third embodiment.

FIG. 9 is a flow chart outlining the operation of the X-ray CT apparatus in the third embodiment.

FIG. 10 is a block diagram illustrating an X-ray CT apparatus in a fourth embodiment.

FIG. 11A is a diagram providing further illustration of a first setter in the fourth embodiment.

FIG. 11B is a diagram providing further illustration of the first setter in the fourth embodiment.

FIG. 11C is a diagram providing further illustration of a second setter in the fourth embodiment.

FIG. 11D is a diagram providing further illustration of the second setter in the fourth embodiment.

FIG. 12A is a diagram providing further illustration of the second setter in the fourth embodiment.

FIG. 12B is a diagram providing further illustration of the second setter in the fourth embodiment.

FIG. 13 is a flow chart outlining the operation of the X-ray CT apparatus in the fourth embodiment.

### MODES FOR CARRYING OUT THE INVENTION

### First Embodiment

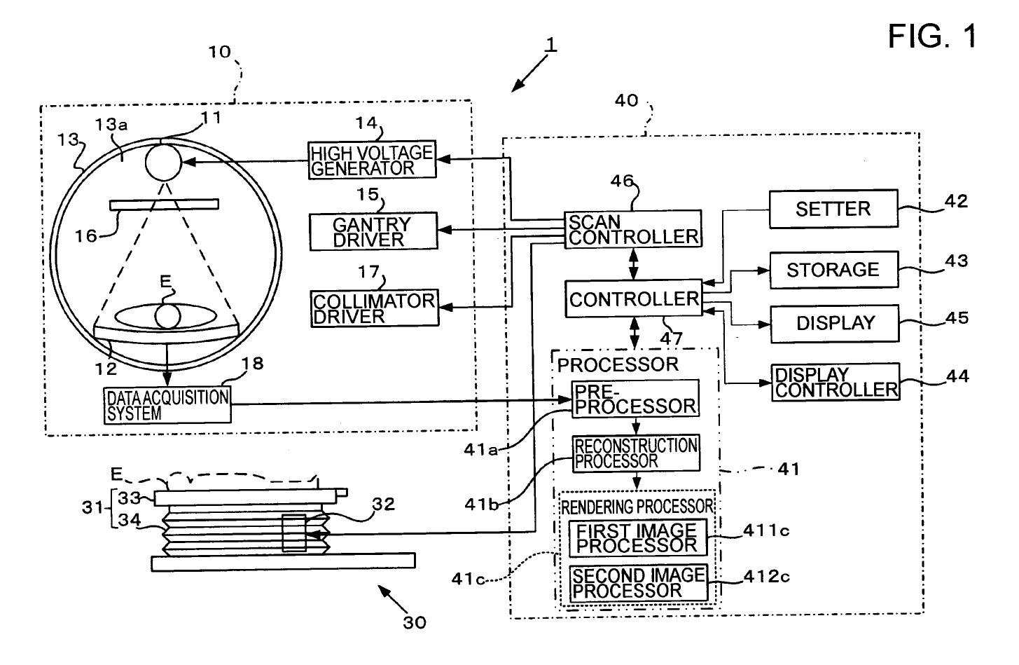

The following is a description of an X-ray CT apparatus 1 according to a first embodiment, with reference to FIG. 1 through FIG. 3. As “image” and “image data” correspond with one another, they are sometimes viewed as the same thing within the present embodiment.

### <Apparatus Configuration>

As depicted in FIG. 1, the X-ray CT apparatus 1 is configured to include a gantry apparatus 10, a couch apparatus 30 and a console device 40.

### [Gantry Apparatus]

The gantry apparatus 10 is an apparatus that irradiates X-rays to a subject E, and acquires detection data in regard to the X-rays passing through the subject E. The gantry apparatus 10 comprises an X-ray generator 11, an X-ray detector 12, a rotator 13, a high voltage generator 14, a gantry driver 15, an X-ray collimator 16, a collimator driver 17 and a data acquisition system 18.

The X-ray generator 11 is configured to include an X-ray tube that generates X-rays (for example, a conical or pyramid-shaped X-ray beam-generating vacuum tube. Not depicted). The X-ray generator 11 irradiates the X-rays to the subject E.

The X-ray detector 12 is configured to include multiple X-ray detection elements (not depicted). The X-ray detector 12 detects X-rays that have passed through the subject E. Specifically, the X-ray detector 12 detects X-ray strength distribution data, which indicates the strength distribution for the X-rays passing through the subject E (hereinafter referred to in some cases as “detection data”) using X-ray detection elements, and outputs this detection data as a current signal. A two-dimensional X-ray detector (plane detector), in which multiple detection elements are arranged in each of two orthogonal directions (slice direction and channel direction), may be used, for example, as the X-ray detector 12. The multiple X-ray detection elements may, for example, be arranged in 320 rows in the slice direction. Using this type of multi-row X-ray detector allows imaging of a three-dimensional area with a width equivalent to the slice direction with a single scan rotation (a volume scan). Here, the slice direction is equivalent to the rostrocaudal direction of the subject E, while the channel direction is equivalent to the rotation direction of the X-ray generator 11.

The rotator 13 is a member to support the X-ray generator 11 and the X-ray detector 12 facing each other so that the subject E is sandwiched therebetween. The rotator 13 has an opening 13a all the way through in the slice direction. The rotator 13 is positioned to rotate in a circular path around the subject E within the gantry apparatus 10. In other words, the X-ray generator 11 and X-ray detector 12 are provided so as to be able to rotate in the circular path around the subject E.

The high-voltage generator 14 applies a high voltage to the X-ray generator 11 (hereinafter, “voltage” refers to the voltage between the anode and cathode of the X-ray tube). The X-ray generator 11 generates X-rays based on this high voltage.

The gantry driver 15 drives the rotation of the rotator 13. The X-ray collimator 16 is provided with a slit (opening) of a specified width, and changes the width of the slit in order to adjust the X-ray fan angle (the angle of spread in the channel direction) and the X-ray cone angle (the angle of spread in the slice direction), of the X-rays output from the X-ray generator 11. The collimator driver 17 drives the X-ray collimator 16 to ensure that the X-rays generated by the X-ray generator 11 are in the specified formation.

The data acquisition system 18 (DAS: Data Acquisition System (DAS)) acquires detection data from the X-ray detector 12 (each of the X-ray detection elements). Furthermore, the data acquisition system 18 converts the acquired detection data (current signal) into a voltage signal, and cyclically integrates and amplifies the voltage signal in order to convert the amplified signal into a digital signal. The data acquisition system 18 transmits the detection data that has been converted into a digital signal to the console device 40. When implementing CT fluoroscopy, the data acquisition system 18 shortens the detection data acquisition rate.

### [Couch Apparatus]

The couch apparatus 30 is an apparatus that places and transfers the subject E for imaging. The couch apparatus 30 comprises a couch 31 and a couch driver 32. The couch 31 comprises a couch top 33 to place the subject E, and a base 34 to support the couch top 33. The couch top 33 can be transferred in the rostrocaudal direction of the subject E and the direction orthogonal thereto, by the couch driver 32. In other words, the couch driver 32 can insert and extract the couch top 33, on which the subject E is placed, into and from the opening 13a of the rotator 13. The base 34 can transfer the couch top 33 vertically (in the direction orthogonal to the rostrocaudal direction of the subject E) by the couch driver 32.

### [Console Device]

The console device 40 is used to input operating instructions to the X-ray CT apparatus 1. Furthermore, the console device 40 has other functions, including that of reconstructing the CT image data (tomographic image data and volume data), which expresses the internal form of the subject E, from the detection data acquired by the gantry apparatus 10. The console device 40 is configured to include a processor 41, a setter 42, a storage 43, a display controller 44, a display 45, a scan controller 46, and a controller 47.

The processor 41 implements various processes in regard to the detection data transmitted from the gantry apparatus 10 (data acquisition system 18). The processor 41 is configured to include a pre-processor 41a, a reconstruction processor 41b and a rendering processor 41c.

The pre-processor 41a implements pre-processing, such as logarithmic conversion, offset correction, sensitivity correction, and beam hardening correction, on the detection data detected by the gantry apparatus 10 (X-ray detector 12), and creates projection data.

The reconstruction processor 41b creates CT image data (tomographic image data and volume data) based on the projection data created by the pre-processor 41a. Reconstruction processing of tomographic image data may involve the application, for example, of an arbitrary method, such as the two-dimensional Fourier conversion method, and the Convolution/Backprojection method. The volume data is generated by interpolation processing of the reconstructed multiple pieces of tomographic image data. Reconstruction processing of the volume data can include, for example, the application of an arbitrary method, such as the cone beam reconstruction method, the multi-slice reconstruction method, and the enlargement reconstruction method. When implementing a volume scan using the aforementioned multi-row X-ray detector, it is possible to reconstruct volume data for a wide area. In addition, when implementing CT fluoroscopy, since the detection data acquisition rate is shortened, the time taken for the reconstruction processor 41b to reconstruct the data is also shortened. As a result, it is possible to create CT image data in real time corresponding to the scan.

The rendering processor 41c implements rendering processing on the volume data created by the reconstruction processor 41b. The rendering processor 41c includes a first image processor 411c and a second image processor 412c.

The first image processor 411c creates a pseudo three-dimensional image (image data) based on volume data. The “pseudo three-dimensional image” is an image that expresses the three-dimensional structure of the subject E in two dimensions. Specifically, by implementing volume rendering processing on the volume data created by the reconstruction processor 41b, the first image processor 411c creates the pseudo three-dimensional image, which is an image (image data) for display use.

The second image processor 412c creates an MPR image (image data) based on volume data. The “MPR image” is an image displaying the required cross-section of the subject E. MPR images include the three orthogonal cross-sections of axial image, sagittal image and coronal image. Alternatively, the second image processor 412c may be used to create an oblique image indicating an arbitrary cross-section as the MPR image. As specific example, the second image processor 412c implements rendering processing at the required angle on the volume data created by the reconstruction processor 41b to create an MPR image.

The setter 42 sets a specified setup image with respect to the image based on volume data. The “setup image” is a required image, drawn onto the image based on volume data. This may, for example, involve drawing a planned puncture needle insertion route for the implementation of a biopsy on the subject E (the route along which the puncture needle is to be inserted, in other words, the planned route) on the image in advance. The image drawn in this way (the image of the planned route) is an example of a setup image. Alternatively, the setup image may be a marked image on which the position of a site of interest (lesion site, and the like) within the image has been marked with a circle or ellipse. The display controller 44 causes the set setup image to be displayed on the image based on volume data. The image based on volume data, on which the setup image is displayed, may be used as a reference image when implementing puncturing, and the like, of the subject E.

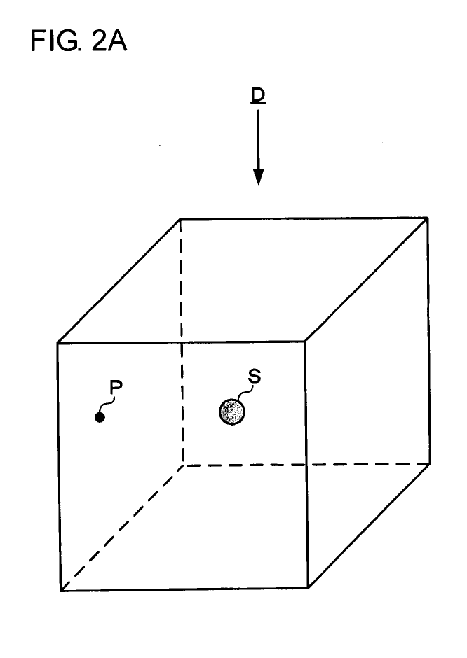

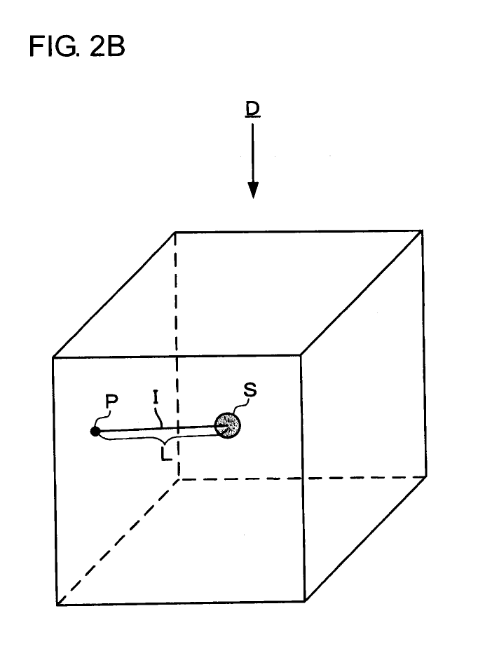

It is described, as specific example of the setter 42, a case in which the image (setup image) is set to depict the planned route on a pseudo three-dimensional image based on volume data (first volume data) obtained from the scan (first scan) implemented at a particular timing. The cube illustrated in FIG. 2A and FIG. 2B is a typical example of a pseudo three-dimensional image D based on volume data. Here, it is assumed that each various surface of the cube represents the body surfaces of the subject E. The display controller 44 causes the display 45 to display the pseudo three-dimensional image D.

The operator uses an input device, and the like, provided on the X-ray CT apparatus 1, and the like, to specify two points of a position S of the target site (lesion site, and the like) to be biopsied, and a position P where the puncture needle is to be inserted on the surface of the body, in regard to the pseudo three-dimensional image D displayed on the display 45 (see FIG. 2A). The setter 42 calculates the shortest distance L between these two points, and sets a line segment connecting this shortest distance L as a setup image I. The display controller 44 causes the set setup image I to be displayed on the pseudo three-dimensional image (see FIG. 2B). Additionally, the setter 42 determines the position (coordinate values. Hereinafter, sometimes referred to as the “setup position”) of the setup image I within the volume data. The setup image I and setup position are stored in the storage 43.

The operator may draw the line segment, and the like, which indicate the planned route directly onto the pseudo three-dimensional image using an input device, and the like. In such a case, the setter 42 sets the relevant drawn line segment as the setup image I. Alternatively, the setter 42 implements image analysis processing using the region growing method, or the like, on volume data to calculate the position of the lesion site and the position on the body surface that is closest to the lesion site. Subsequently, the setter 42 may calculate the line segment connecting these two points to set the line segment as the setup image I.

The storage 43 is configured by comprising a semiconductor storing device such as RAM, ROM, and the like. The storage 43 stores not only setup images and setup image setup positions, but also detection data, projection data, or alternatively CT image data subsequent to reconstruction processing, and the like.

The display controller 44 implements various controls relating to image display. For example, the display controller 44 controls the display on the display 45 of the pseudo three-dimensional image created by the first image processor 411c, the MPR image (axial image, sagittal image, coronal image or oblique image) created by the second image processor 412c, and the like.

Furthermore, in the present embodiment, the display controller causes the setup image to be displayed in the position corresponding to the setup position in the image based on the volume data displayed on the display 45.

It is described, as a specific example of the display controller 44 causing the display 45 to display a pseudo three-dimensional image based on the volume data (second volume data) obtained through a scan (second scan) implemented at a timing different from that of the first scan. In the present embodiment, the first volume data and the second volume data are based on the same number of sets of tomographic image data, and have the same number of pixels within the images. Additionally, it is assumed that the imaging conditions for the first scan and the second scan (imaging positions, rotation speed of the rotator 13, and the like) are the same. In other words, it is assumed that the first volume data and the second volume data are on the same coordinate values system.

In this case, the display controller 44 causes the same image as the setup image to be displayed in the position corresponding to the setup position stored in the storage 43. The display controller 44 may replace the pixels (pixel value) of the pseudo three-dimensional image based on the second volume data with the pixels (pixel value) of the setup image, as the display format of the setup image. Alternatively, the display controller 44 may also superimpose the setup image onto the pseudo three-dimensional image based on the second volume data. The image based on the second volume data, on which the setup image is displayed, may be used as a new reference image.

The display 45 is configured by an arbitrary display device such as an LCD (Liquid Crystal Display), CRT (Cathode Ray Tube) display, or the like. The display 45, for example, displays the MPR image obtained by rendering the volume data.

The scan controller 46 controls the various operations relating to X-ray scanning. The scan controller 46, for example, controls the high voltage generator 14 to apply a high voltage to the X-ray generator 11. The scan controller 46 controls the gantry driver 15 to rotate the rotator 13. The scan controller 46 controls the collimator driver 17 to operate the X-ray collimator 16. The scan controller 46 also controls the couch driver 32 to transfer the couch 31.

The controller 47 controls the movement of the gantry apparatus 10, couch apparatus 30 and console device 40, thereby controlling the X-ray CT apparatus 1 as a whole. For example, the controller 47 controls the scan controller 46, thereby causing the gantry apparatus 10 to implement the preparatory scan and the main scan, and to acquire detection data. Furthermore, the controller 47 controls the processor 41, thereby causing the processor 41 to implement various types of processing on the detection data (pre-processing, reconstruction processing, etc.) Alternatively, the controller 47 controls the display controller 44, thereby causing the display controller 44 to display the image on based on the CT image data, stored in the storage 43, on the display 45.

### <Operation>

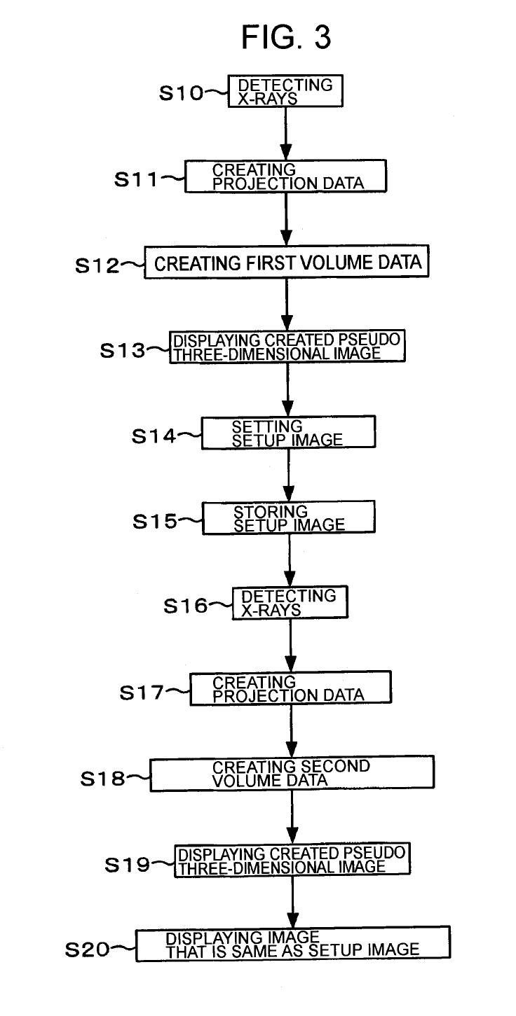

Next, there follows a description of the operation of the X-ray CT apparatus 1 in the present embodiment, in reference to FIG. 3. The explanation given here is of a case in which biopsy is carried out using CT fluoroscopy, once the planned route of the puncture needle has been created.

Prior to beginning the biopsy, the X-ray CT apparatus 1 first implements an X-ray scan (first scan) on the subject E, and creates volume data (first volume data).

Specifically, the X-ray generator 11 irradiates the subject E with X-rays. The X-ray detector 12 detects the X-rays passing through the subject E, and obtains detection data (S10). The detection data detected by the X-ray detector 12 is acquired by the data acquisition system 18, before being transmitted to the processor 41 (pre-processor 41a).

The pre-processor 41a implements logarithmic conversion, offset correction, sensitivity correction, beam hardening correction and other pre-processes on the detection data obtained in S10, and creates projection data (S11). The projection data created is transmitted to the reconstruction processor 41b based on the control of the controller 47.

The reconstruction processor 41b creates multiple sets of tomographic image data based on the projection data created in S11. Furthermore, the reconstruction processor 41b creates the first volume data by interpolation processing of the multiple sets of tomographic image data (S 12).

The first image processor 411c creates a pseudo three-dimensional image by rendering the first volume data created in S12. The display controller 44 causes the display 45 to display the created pseudo three-dimensional image (S 13).

The operator plans a puncture needle insertion route (planned route) in reference to the pseudo three-dimensional image displayed on the display 45. The operator specifies the position of the lesion site and the insertion position of the puncture needle within the pseudo three-dimensional image using an input device, and the like. The setter 42 sets the line segment connecting the specified positions as a setup image (S14). The display controller 44 causes the set setup image to be displayed on the pseudo three-dimensional image. The setter 42 transmits the setup image and setup image coordinate values (setup position) to the storage 43. The storage 43 stores the setup image and the relevant coordinate values (setup position) (S15).

Subsequently, the operator begins the biopsy on the subject E, while referring to the pseudo three-dimensional image on which the setup image is displayed.

When the biopsy has progressed to a certain extent (once the puncture needle has been inserted into the subject E), the X-ray CT apparatus 1 once more implements an X-ray scan (second scan) on the subject E in order to confirm the state of the puncture (whether or not the puncture needle is following the planned route, and the like), and creates volume data (second volume data).

In other words, similarly to the first scan, the X-ray generator 11 irradiates the subject E with X-rays. The X-ray detector 12 detects the X-rays passing through the subject E, and obtains detection data (S16). As stated above, the first scan and the second scan are implemented under the same imaging conditions.

The pre-processor 41a implements pre-processing on the detection data obtained in S16, and creates projection data (S 17). The reconstruction processor 41b implements interpolation processing on the multiple sets of tomographic image data created based on the projection data created in S17, to create the second volume data (S18). The first image processor 411c renders the second volume data created in S18 to create a pseudo three-dimensional image (S19).

The display controller 44 causes the display 45 to display the pseudo three-dimensional image created in S19, as well as causing an image that is the same as the setup image set in S14 to be displayed in the position corresponding to the setup position that is stored in S15 in the pseudo three-dimensional image based on the second volume data (S20).

In this way, by causing the display of the setup image (the image depicting the planned route) pre-drawn before the biopsy begins, on the image based on the second volume data, it is possible to easily ascertain the setup image, even within the image based on the volume data (second volume data), which is different from the volume data (first volume data) in which the setup image is set. Furthermore, as the biopsy progresses, if the puncture needle becomes misaligned with the planned route, the misalignment between the position of the puncture needle depicted in the image based on volume data and the setup image displayed thereon will be displayed. On the other hand, if the puncture needle is inserted in line with the planned route, the position of the puncture needle depicted in the image based on volume data and the setup image displayed thereon will be displayed as overlapping. In other words, by referring to the image on which the setup image is displayed, the operator can easily ascertain any misalignment of the puncture needle (misalignment from the planned route).

The processor 41, setter 42, display controller 44, scan controller 46 and controller 47 may be configured from processing apparatus not depicted, such as a CPU (Central Processing Unit), GPU (Graphic Processing Unit) or ASIC (Application Specific Integrated Circuit), and storing device not depicted, such as a ROM (Read Only Memory), RAM (Random Access Memory) or HDD (Hard Disc Drive). The storing device stores processing programs that enable the processor 41 to implement its functions. Further, the storing device stores programs for the setter processing that enable the setter 42 to implement its functions. Additionally, the storing device stores display control programs that enable the display controller 44 to implement its functions. Furthermore, the storing device stores scan control programs that enable the scan controller 46 to implement its functions. The storing device also stores control programs that enable the controller 47 to implement its functions. The processing apparatus, such as CPU, implements functions of the various devices by implementing each program stored in the storage apparatus.

To this point, the configuration and operation of the single X-ray CT apparatus 1 has been described. The configuration of the present embodiment, however, may be realized as an X-ray CT system including the X-ray CT apparatus 1.

For example, the X-ray CT apparatus 1 sets a setup image in an image based on pre-created volume data, and stores the setup image and setup image setup position. Next, a CT fluoroscopy biopsy is done using another X-ray CT apparatus. In this case, the other X-ray CT apparatus causes the display to display the image based on the second volume data obtained by CT fluoroscopy. Furthermore, the other X-ray CT apparatus reads the stored setup image and setup image setup position from the X-ray CT apparatus 1, and causes the setup image to be displayed in the position corresponding to the relevant setup image setup position within the image based on the second volume data.

Alternatively, the X-ray CT apparatus 1 may be used to create an image based on the first volume data. A computer that is separate to the X-ray CT apparatus 1 sets the setup image in the image based on the first volume data, and stores the setup image and setup image setup position. Next, if the X-ray CT apparatus 1 (or another X-ray CT apparatus) is to be used to implement CT fluoroscopy, the X-ray CT apparatus 1 causes the display to display the image based on the second volume data, obtained by CT fluoroscopy. Furthermore, the X-ray CT apparatus 1 may read the stored setup image and setup image setup position from the computer, and cause the setup image to be displayed in the position corresponding to the relevant setup image setup position on the image based on the second volume data.

### <Operation and Effect>

The following is a description of the operation and effect of the present embodiment.

The X-ray CT apparatus 1 in the present embodiment creates first volume data and second volume data based on the results of scanning a subject with X-rays at different timings. The X-ray CT apparatus 1 comprises a setter 42, a storage device 43, and a display controller 44. The setter 42 sets a specified setup image in regard to the image based on the first volume data. The storage 43 stores the setup image and the setup position of the setup image. The display controller 44 causes the image based on the second volume data to be displayed on the display 45, as well as causing the display of the setup image in the position corresponding to the setup position in the image based on the second volume data.

Specifically, the X-ray CT apparatus 1 has a first image processor 411c. The first image processor 411c creates a pseudo three-dimensional image that expresses the three-dimensional structure of a subject E in two dimensions, based on the volume data. The setter 42 sets a setup image in regard to the pseudo three-dimensional image based on the first volume data. The display controller 44 causes the display 45 to display the pseudo three-dimensional image based on the second volume data, as well as causing the setup image to be display in the position corresponding to the setup position in the pseudo three-dimensional image based on the second volume data.

In addition, the configuration of the present embodiment may be realized as an X-ray CT system. The X-ray CT system comprises at least one X-ray CT apparatus, a setter 42, a storage device 43 and a display controller 44. The X-ray CT apparatus creates volume data based on the results of scanning a subject E with X-rays. The setter 42 sets a specified setup image in regard to the image based on a pre-created first volume data. The storage device 43 stores the setup image and the setup image setup position. The display controller 44 causes a newly created image based on the second volume data to be displayed on a display 45, as well as causing the setup image to be displayed in the position corresponding to the setup position in the image based on the second volume data.

In this way, the display controller 44 allows the set setup image in regard to the pseudo three-dimensional image based on the first volume data to be displayed in the position corresponding to the setup position in the pseudo three-dimensional image based on the second volume data. For example, in a biopsy using CT fluoroscopy, the display controller 44 can display the image depicting a pre-specified planned route in the same position, even on the pseudo three-dimensional image based on the volume data (second volume data) obtained each time the X-ray scan is performed. As a result, the operator can confirm the planned route in the current image by referring to the pseudo three-dimensional image. Furthermore, if the puncture needle is depicted in the image based on the second volume data, the operator can also see any misalignment between the puncture needle and the planned route, thereby easily ascertaining whether or not the puncture needle is proceeding according to the planned route. In other words, according to the present embodiment, it is possible to easily recognize the pre-specified image (setup image) on the image obtained at the current time.

### Second Embodiment

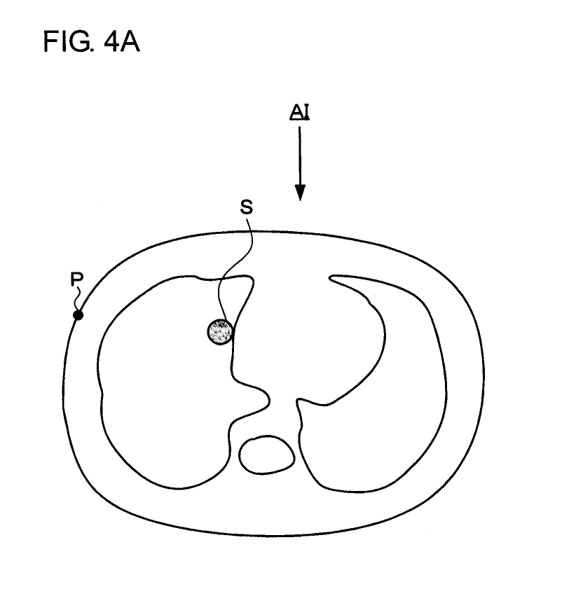

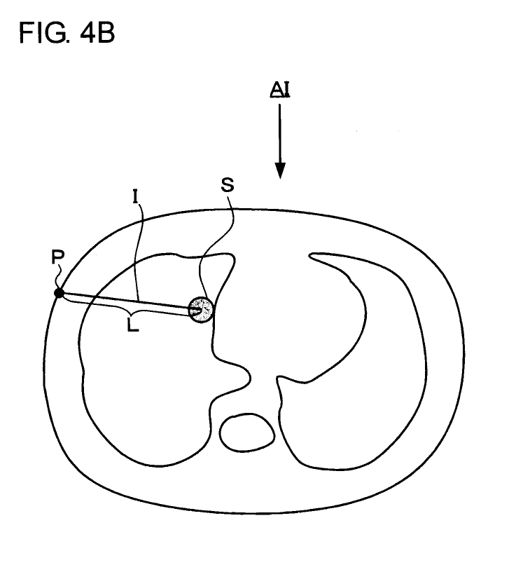

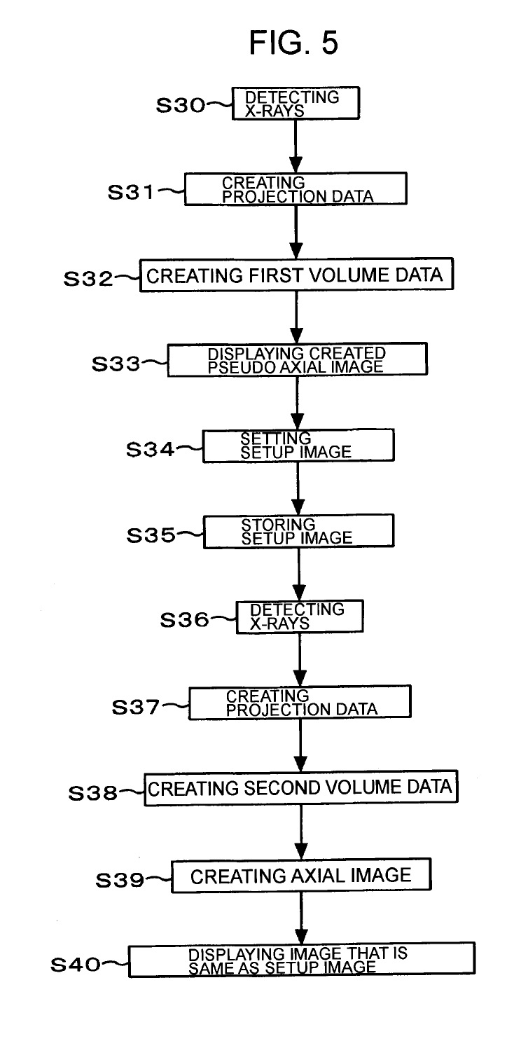

The following is a description of the X-ray CT apparatus 1 according to a second embodiment, in reference to FIG. 4A through FIG. 5. In the present embodiment, the setter 42 sets a setup image in relation to the MPR image based on the first volume data. Next, there follows a description of the configuration of the display controller 44, which causes the display of the relevant setup image on the MPR image based on the second volume data. No details are given in regard to configurations that are the same as those in the first embodiment. The following description uses an axial image as an example of the MPR image, however, the present embodiment configuration may be applied in the same way with either a sagittal image or a coronal image.

The setter 42 in the present embodiment sets a specified setup image in regard to the MPR image based on volume data. The MPR image is created by a second image processor 412c.

As specific example of the setter 42, it is described a case of setting an image (setup image) depicting a planned route for a puncture needle in regard to an axial image based on volume data (the first volume data) obtained from the scan (first scan) implemented at a certain timing. FIG. 4A and FIG. 4B depict an axial image AI based on the volume data. The display controller 44 causes the display 45 to display the axial image AI.

The operator uses an input device, and the like, to specify two points of a position S of the target site (lesion site, and the like) to be biopsied, and a position P where the puncture needle is to be inserted on the surface of the body, in regard to the axial image AI displayed on the display 45 (see FIG. 4A). The setter 42 calculates a shortest distance L between these two points, and sets a line segment connecting this shortest distance L as a setup image I. The display controller 44 causes the display of the set setup image I on the axial image AI (see FIG. 4B). Additionally, the setter 42 calculates the setup position (coordinate values) within the axial image AI. The setup image I and setup position are stored in the storage 43. The axial image AI is an image based on three-dimensional volume data. As a result, the position of the set setup image within the axial image AI can be specified with three-dimensional coordinate values.

In the present embodiment, the display controller 44 causes to the setup image to be displayed in the position corresponding to the setup position in the MPR image based on the volume data displayed by the display 45.

As specific example of the display controller 44, it is described a case that the display controller 44 causes the display 45 to display the axial image based on volume data (second volume data) obtained from the scan (second scan) implemented at a different timing to the first scan. It is assumed that the axial image based on the first volume data and the axial image based on the second volume data depict a cross-section at the same position in the rostrocaudal direction.

In this case, the display controller 44 causes an image that is the same as the setup image to be displayed in the position within the axial image that corresponds to the setup position stored in the storage 43.

Alternatively, similarly to the first embodiment, the display controller 44 may cause an image that is the same as the setup image to be displayed in the position corresponding to the setup position within the pseudo three-dimensional image based on the second volume data. As mentioned above, the setup position set in regard to the MPR image (axial image) based on the first volume data has three-dimensional coordinate values. As a result, even if the image based on the second volume data is a pseudo three-dimensional image, it is possible to identify the position corresponding to the setup position.

### <Operation>

The following is a description of the operation of the X-ray CT apparatus 1 in the present embodiment, in reference to FIG. 5. Here, the description is of the operation wherein a biopsy is implemented using CT fluoroscopy, after creating the planned route for the puncture needle within the axial image.

Prior to beginning the biopsy, the X-ray CT apparatus 1 first carries out an X-ray scan (first scan) of a subject E and creates volume data (first volume data).

Specifically, the X-ray generator 11 irradiates the subject E with X-rays. The X-ray detector 12 detects X-rays passing through the subject E, and obtains detection data (S30). The pre-processor 41a implements pre-processing, such as logarithmic conversion, offset correction, sensitivity correction, and beam hardening correction, on the detection data obtained in S30, and creates projection data (S31). The reconstruction processor 41b creates multiple sets of tomographic image data based on the projection data created in S31. Furthermore, the reconstruction processor 41b creates the first volume data by interpolation processing of the multiple sets of tomographic image data (S32).

The second image processor 412c creates an axial image by rendering the first volume data created in S32. The display controller 44 causes the display 45 to display the created axial image (S33).

The operator plans a puncture needle insertion route (planned route) in reference to the axial image displayed on the display 45. The operator specifies the position of the lesion area and the insertion position of the puncture needle within the axial image using an input device, and the like. The setter 42 sets a line segment connecting the specified positions as setup image (S34). The display controller 44 causes the display of the set setup image on the axial image. The setter 42 transmits the setup image coordinate values (setup position) to the storage 43. The storage 43 stores the setup image and relevant coordinate values (setup position) (S35).

Subsequently, the operator begins puncturing on the subject E, while referring to the axial image on which the setup image is displayed.

When the biopsy has progressed to a certain extent (once the puncture needle has been inserted into the subject E), the X-ray CT apparatus 1 implements an X-ray scan (second scan) on the subject E again in order to confirm the state of the puncture (whether or not the puncture needle is following the planned route, and the like), and creates volume data (second volume data).

In other words, similarly to the first scan, the X-ray generator 11 irradiates the subject E with X-rays. The X-ray detector 12 detects the X-rays passing through the subject E, and obtains detection data (S36). As in the first embodiment, the first scan and the second scan are implemented under the same imaging conditions.

The pre-processor 41a implements pre-processing on the detection data obtained in S36, and creates projection data (S37). The reconstruction processor 41b implements interpolation processing on the multiple sets of tomographic image data created based on the projection data created in S37, to create the second volume data (S38). The second image processor 412c renders the second volume data to create an axial image (S39).

The display controller 44 causes the display 45 to display the axial image created in S39, as well as causing the display of an image that is the same as the setup image set in S34 in the position corresponding to the setup position stored in S35, in the axial image based on the second volume data (S40).

### <Operation and Effect>

The following is a description of the operation and effect of the present embodiment.

The X-ray CT apparatus 1 in the present embodiment has a second image processor 412c. The second image processor 412c creates an MPR image depicting a cross-section of a subject E, based on volume data. The setter 42 sets a setup image in regard to the MPR image based on the first volume data. The display controller 44 causes the display 45 to display the MPR image based on the second volume data, as well as causing the setup image to be displayed in the position corresponding to the setup position in the MPR image based on the second volume data.

In addition to this, the X-ray CT apparatus 1 in the present embodiment includes a first image processor 411c and a second image processor 412c. The first image processor 411c creates a pseudo three-dimensional image that expresses the three-dimensional structure of the subject E in two dimensions, based on volume data. The second image processor 412c creates an MPR image depicting a cross-section of the subject E, based on volume data. The setter 42 sets a setup image in regard to the MPR image based on the first volume data. The display controller 44 causes the display 45 to display the pseudo three-dimensional image based on the second volume data, as well as causing the setup image to be displayed in the position corresponding to the setup position in the pseudo three-dimensional image based on the second volume data.

Furthermore, the second image processor 412c in X-ray CT apparatus 1 of the present embodiment creates at least one out of an axial image, a sagittal image, a coronal image and an oblique image of the subject E as the MPR image.

In this way, the display controller 44 allows the setup image set in regard to the MPR image based on the first volume data to be displayed in the position corresponding to the setup position in the image (pseudo three-dimensional image or MPR image) based on the second volume data. For example, in a biopsy using CT fluoroscopy, the display controller 44 can display the image depicting a pre-specified planned route in the same position on the image based on the volume data (second volume data) obtained each time the X-ray scan is performed. As a result, the operator can confirm the planned route in the current image by referring to this image. Furthermore, if the puncture needle is depicted in the image based on the second volume data, the operator can also see any misalignment between the puncture needle and the planned route, thereby easily ascertaining whether or not the puncture needle is proceeding according to the planned route. In other words, according to the present embodiment, it is possible to easily recognize the pre-specified image (setup image) on the image obtained at the current time. Furthermore, the setup image can easily be set using a two-dimensional MPR image.

### Modified Example 1

In the second embodiment, a setup image was specified in regard to the axial image. Here, since the setup image is specified from an image based on volume data, the setup image possesses three-dimensional coordinate values. As a result, the setter 42 can also automatically set the setup image in the position corresponding to the relevant three-dimensional coordinate values within a coronal image or sagittal image created from the volume data that is the source of the axial image.

In other words, the setter 42 can set a setup image in regard to an MPR image depicting a particular cross-section, while at the same time setting, based on the setup image setup position, a setup image in regard to an MPR image depicting another cross-section. The display controller 44 causes the display of the set setup image on each MPR image.

### Modified Example 2

By observing the cross-sectional image in line with the image (setup image) depicting the planned route for the puncture needle, set by using the setter 42, the operator is able to ascertain the entire planned route on the two-dimensional image. In this case, the second image processor 412c creates an oblique image of the cross section in line with the setup image, based on the first volume data.

Furthermore, the second image processor 412c can also store the cross-section position of the cross-sectional oblique image in line with the setup image, and create the same cross-sectional oblique image within the second volume data. In other words, the second image processor 412c constantly creates an oblique image at the same cross-section position in regard to each set of volume data (the first to the nth volume data) obtained at different timings. The created oblique images are displayed on the display 45 by the display controller 44.

Here, for example, if the puncture needle is not following the planned route, the puncture needle will not be depicted on the oblique image based on the second volume data. As a result, the operator can easily ascertain the misalignment of the puncture needle (misalignment from the planned route). The image created by the second image processor 412c is not restricted to an oblique image, but may be any cross-sectional image in line with the setup image. For example, if the insertion route is planned orthogonally to the rostrocaudal direction of the subject E, the image created by the second image processor 412c should ideally be an axial image.

### Modified Example 3

Furthermore, after a biopsy is carried out in regard to the subject E, in some cases the operator may wish to confirm the route by which the puncture needle actually progressed (the route by which the puncture needle was inserted). In this case, it is desirable for a cross-section including the puncture needle to be created and stored for each of the volume data (the first to the nth volume data) obtained at different timings.

As follows, the configuration of this modified example will be set forth, in which the puncture needle position is detected in each set of volume data, to create a new image at the cross-section that includes the puncture needle. A description is given below in which an oblique image is created as a new image.

For example, the processor 41 identifies the position of the puncture needle in regard to each of multiple sets of volume data. Specifically, the processor 41 takes the difference between the tomographic image data configuring the volume data in regard to each of multiple sets of volume data, and identifies the tomographic image data in which the difference is largest. The processor 41 then implements image processing of the identified tomographic image data to detect the edges, and the like, and identifies the puncture needle position. The identification of the puncture needle position within the volume data may be done not only by the aforementioned method but also by any known method.

The second image processor 412c renders the volume data in a specified direction based on the identified puncture needle position, to create an oblique image, which is a cross-section including the puncture needle. The second image processor 412c implements this process for each of multiple sets of volume data. Thus, the oblique image created by the second image processor 412c identifies always has the puncture needle displayed therein. The oblique image created by the second image processor 412c is stored in the storage 43. As a result, the operator may observe the multiple oblique images stored in the storage 43 after the biopsy is completed to check once again the route by which the puncture needle actually progressed (the route by which the puncture needle was inserted).

<Common Effects within the First Embodiment and Second Embodiment>

Of the first embodiment and second embodiment outlined above, in the X-ray CT apparatus in at least one of the embodiments, the display controller can cause the display of the set up image set in regard to the image based on the first volume data in the position corresponding to the setup position within the image based on the second volume data. In other words, according to the present embodiments, the predetermined image (setup image) can be easily recognized on an image obtained at the current time.

### Third Embodiment

There are some cases, for example, in which the effect of movement of the subject or the skill level of the doctor in regard to the use of puncture needles makes it difficult for the puncture needle to be inserted according to the planned route. In other words, the planned route and the actual position (route) of the puncture needle may become misaligned, and effect an impediment to an accurate biopsy. On the other hand, how the puncture needle insertion position and direction should be corrected in regard to the misalignment of the puncture needle from the planned route depends largely on the experience of the doctor, and the like.

The present embodiment is designed to solve the aforementioned problems, with the objective of providing a technique that facilitates the display of an image reflecting the misalignment between the planned route and the puncture needle.

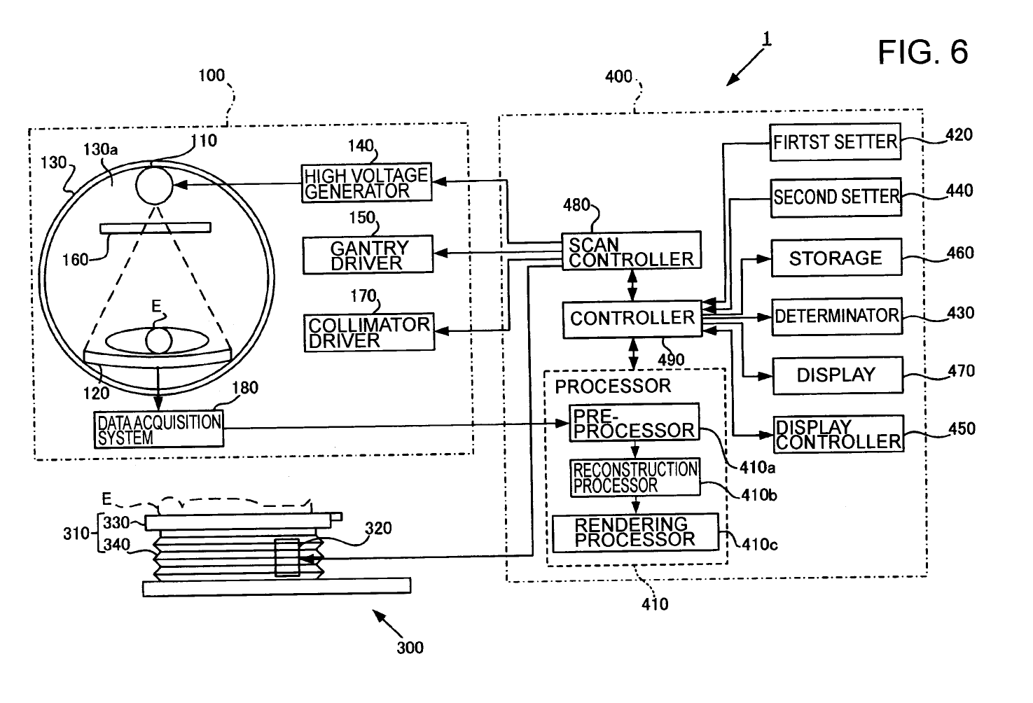

The following is a description of the X-ray CT apparatus 1 in a third embodiment, in reference to FIG. 6 through FIG. 9.

### <Apparatus Configuration>

As depicted in FIG. 6, the X-ray CT apparatus 1 is configured to include a gantry apparatus 100, a couch apparatus 300 and a console device 400.

### [Gantry Apparatus]

The gantry apparatus 100 is an apparatus that irradiates a subject E with X-rays, and acquires detection data in regard to the X-rays passing through the subject E. The gantry apparatus 100 comprises an X-ray generator 110, an X-ray detector 120, a rotator 130, a high voltage generator 140, a gantry driver 150, an X-ray collimator 160, a collimator driver 170 and a data acquisition system 180.

The X-ray generator 110 is configured to include an X-ray tube that generates X-rays (for example, a conical or pyramid-shaped beam-generating vacuum tube. Not depicted). The X-ray generator 110 irradiates the generated X-rays to the subject E.

The X-ray detector 120 is configured to include multiple X-ray detection elements (not depicted). The X-ray detector 120 detects the X-rays that have passed through the subject E. Specifically, the X-ray detector 120 detects X-ray strength distribution data (detection data), which indicates the strength distribution for the X-rays passing through the subject E using X-ray detection elements, and outputs this detection data as a current signal. For example, as the X-ray detector 120, a two-dimensional X-ray detector (plane detector), in which multiple detection elements are arranged in each of two orthogonal directions (slice direction and channel direction), may be used. The multiple X-ray detection elements, for example, are arranged in 320 rows in the slice direction. Using this type of multi-row X-ray detector allows imaging of a three-dimensional imaging area with a width equivalent to the slice direction with a single scan rotation (a volume scan). Here, the slice direction is equivalent to the rostrocaudal direction of the subject E, while the channel direction is equivalent to the rotation direction of the X-ray generator 110.

The rotator 130 is a member to support the X-ray generator 110 and the X-ray detector 120 facing each other so that the subject E is sandwiched therebetween. The rotator 130 has an opening 130a all the way through in the slice direction. The rotator 130 is positioned to rotate in a circular path around the subject E within the gantry apparatus 100. In other words, the X-ray generator 110 and X-ray detector 120 are provided so as to be able to rotate in the circular path around the subject E.

The high-voltage generator 140 applies a high voltage to the X-ray generator 110. The X-ray generator 110 generates X-rays based on this high voltage.

The gantry driver 150 rotatably drives the rotator 130. The X-ray collimator 160 is provided with a slit (opening) of a specified width, and changes the width of the slit in order to adjust the X-ray fan angle (the angle of spread in the channel direction) and the X-ray cone angle (the angle of spread in the slice direction), of the X-rays output from the X-ray generator 110. The collimator driver 170 drives the X-ray collimator 160 to ensure that the X-rays generated by the X-ray generator 110 are in the specified formation.

The data acquisition system 180 (DAS) acquires detection data from the X-ray detector 120 (each of the X-ray detection elements). Furthermore, the data acquisition system 180 converts the acquired detection data (current signal) into a voltage signal, and cyclically integrates and amplifies the voltage signal in order to convert the amplified voltage signal into a digital signal. The data acquisition system 180 transmits the detection data that has been converted into a digital signal to the console device 400. When implementing CT fluoroscopy, the data acquisition system 180 shortens the detection data acquisition rate.

### [Couch Apparatus]

The couch apparatus 300 is an apparatus that places and transfers the subject E for imaging. The couch apparatus 300 comprises a couch 310 and a couch driver 320. The couch 310 comprises a couch top 330 to place the subject E and a base 340 to support the couch top 330. The couch top 330 can be transferred, in the rostrocaudal direction of the subject E and the direction orthogonal thereto, by the couch driver 320. In other words, the couch driver 320 can insert and extract the couch top 330, on which the subject E is placed, into and from the opening 130a of the rotator 130. The base 340 can transfer the couch top 330 vertically (in the direction orthogonal to the body axis of subject E) by the couch driver 320.

### [Console Device]

The console device 400 is used to input operating instructions to the X-ray CT apparatus 1. Furthermore, the console device 400 has other functions, including that of reconstructing the CT image data (tomographic image data and volume data), which expresses the internal form of the subject E, from the detection data acquired by the gantry apparatus 100. The console device 400 is configured to include a processor 410, a first setter 420, a determinator 430, a second setter 440, a display controller 450, a storage 460, a display 470, a scan controller 480, and a controller 490.

The processor 410 implements various processes in regard to the detection data transmitted from the gantry apparatus 100 (data acquisition system 180). The processor 410 is configured to include a pre-processor 410a, a reconstruction processor 410b and a rendering processor 410c.

The pre-processor 410a implements pre-processing, such as logarithmic conversion, offset correction, sensitivity correction, and beam hardening correction, on the detection data detected by the gantry apparatus 100 (X-ray detector 120), and creates projection data.

The reconstruction processor 410b creates CT image data (tomographic image data and volume data) based on the projection data created by the pre-processor 410a. Reconstruction processing of tomographic image data may involve the application, for example, of an arbitrary method, such as the two-dimensional Fourier conversion method, and the convolution/back projection method. The volume data is generated by interpolation processing of the reconstructed multiple pieces of tomographic image data. Reconstruction processing of the volume data can include, for example, the application of arbitrary method, such as the cone beam reconstruction method, the multi-slice reconstruction method, and the enlargement reconstruction method. When implementing a volume scan using the aforementioned multi-row X-ray detector, it is possible to reconstruct volume data for a wide area. In addition, when implementing CT fluoroscopy, since the detection data acquisition rate is shortened, the time taken for the reconstruction processor 410b to reconstruct the data is also shortened. As a result, it is possible to create CT image data in real time corresponding to the scan.

The rendering processor 410c implements rendering processing on the volume data created by the reconstruction processor 410b.

The rendering processor 410c, for example, creates a pseudo three-dimensional image (image data) by volume rendering volume data. The “pseudo three-dimensional image” is an image that expresses the three-dimensional structure of the subject E in two dimensions.

Furthermore, the rendering processor 410c creates an MPR image (image data) by rendering volume data in the required direction. The “MPR image” is an image displaying the required cross-section of the subject E. MPR images include the three orthogonal cross-sections of axial image, sagittal image and coronal image. Alternatively, the rendering processor 410c may create an oblique image indicating an arbitrary cross-section as the MPR image.

The first setter 420 is used to set the puncture needle insertion route in the subject E on the image based on the pre-created volume data. The pre-created volume data refers to the volume data obtained from the X-ray scan implemented at the stage prior to the implementation of the biopsy.

The insertion route set by the first setter 420 is the route (planned route) indicating the route by which the puncture needle is to be inserted into the subject E. The insertion route corresponds to the image of the insertion route displayed on the display 470, therefore, in some cases, hereinafter, the insertion route and the image thereof are considered to be the same thing.

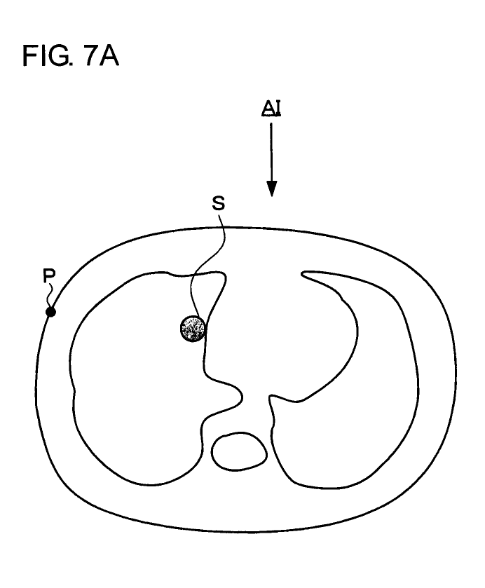

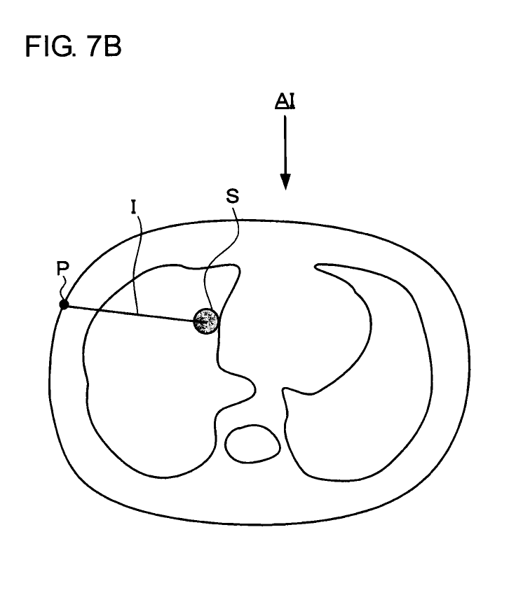

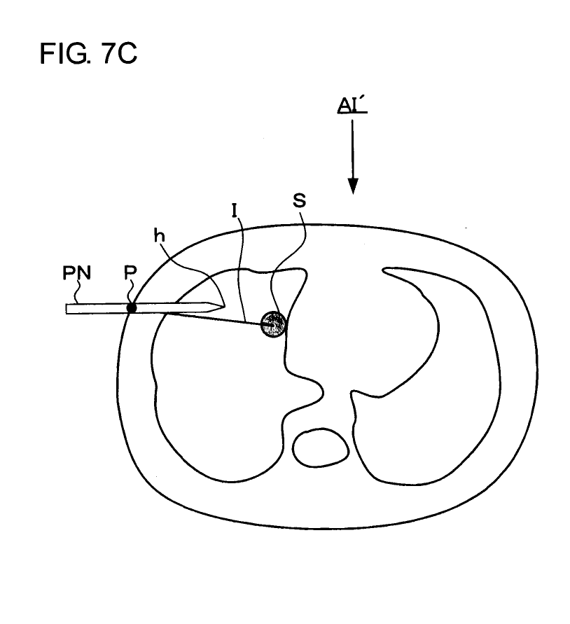

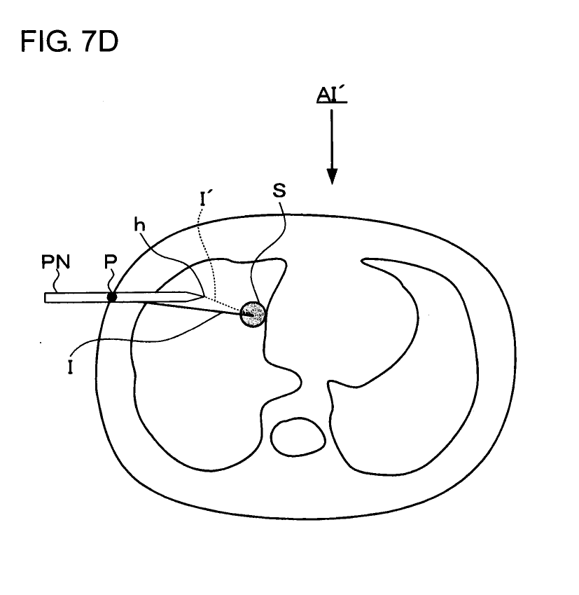

As a specific example of the first setter 420, it is described a case that a puncture needle insertion route (planned route) is set in regard to an axial image AI based on volume data (the first volume data) obtained from the scan (first scan) implemented at a certain timing. FIG. 7A and FIG. 7B depict the axial image AI based on the volume data. The display controller 450 causes the display 470 to display the axial image AI.

The operator uses an input device, and the like provided on X-ray CT apparatus 1, and the like to specify two points of a position S of the target site (lesion site, and the like) to be biopsied, and a position P where the puncture needle is to be inserted, in regard to the axial image AI displayed on the display 470 (see FIG. 7A). The first setter 420 calculates a shortest distance L between these two points on the axial image AI, and sets a line segment forming this shortest distance L as insertion route I. The display controller 450 causes the display of the set insertion route I on the axial image AI (see FIG. 7B). Additionally, the first setter 420 determines the position (coordinate values) of the insertion route I within the axial image AI. The insertion route I image and insertion route I position are stored in the storage 460. The axial image AI is an image based on three-dimensional volume data. As a result, the position of the insertion route I set within the axial image AI can be specified using three-dimensional coordinate values.

The operator may draw the line segment depicting the insertion route I on the axial image AI directly (manually), using an input device, and the like. In this case, the first setter 420 sets the relevant drawn line segment as the insertion route I. Alternatively, the first setter 420 implements image analysis processing such as edge detection and the like of the axial image AI to calculate the position S of the lesion site and the position on the surface of the body closest to the lesion site. The first setter 420 can then calculate the line segment connecting these points, and sets the line segment (automatically) as the insertion route I.

Furthermore, the image used to set the insertion route I is not limited an axial image AI. The first setter 420 may set the insertion route I in regard to either a sagittal image or a coronal image, using the same methods. Alternatively, the first setter 420 may set the insertion route I in regard to the pseudo three-dimensional image based on the volume data (the image depicting the three-dimensional structure of the subject E in two-dimensions).

The determinator 430 determines whether any misalignment has occurred between the puncture needle and the insertion route within the image based on volume data created based on the results of the scan implemented when the puncture needle is inserted into the subject E. “Misalignment” is any difference in position between the set insertion route position and the puncture needle position when the puncture needle is inserted into the subject E. Misalignment is expressed, for example, as the distance from the position of the puncture needle tip to the set insertion route. In other words, if there is no misalignment (the puncture is implemented according to the insertion route) the relevant distance will be 0. Alternatively, the “misalignment” may be expressed as the angle between the set insertion route and the puncture needle (in which case, if there is no misalignment, the relevant angle will be 0).

As a specific example of the determinator 430, there follows a description of a case in which the first setter 420 sets the insertion route I in regard to the axial image AI based on the first volume data.

The rendering processor 410c creates an axial image AI′ based on the volume data (second volume data) obtained the scan (second scan) implemented at a different timing to the first scan (while the puncture needle is inserted in the subject E). The determinator 430 reads the position (coordinate values) of the insertion route I set by the first setter 420 from the storage 460. Furthermore, the determinator 430 detects, in the axial image AI′, a tip position h (coordinate values) of a puncture needle PN inserted into the subject E by image processing, such as edge detection. The determinator 430 then determines whether or not the tip position h of the puncture needle PN is in line with the set insertion route I.

If the tip position h of the puncture needle PN is in line with the set insertion route I (the coordinate values of the tip position h of the puncture needle are included within the coordinate values of the insertion route I), the determinator 430 determines that there is no misalignment. On the other hand, if the tip position h of the puncture needle PN is not in line with the set insertion route I (the coordinate values of the tip position h of the puncture needle are not included within the coordinate values of the insertion route I), the determinator 430 determines that there is misalignment. The determinator 430 can also detect the difference between the insertion route I and the tip position h of the puncture needle PN as the extent of misalignment.

In the present embodiment, first volume data and the second volume data are based on the same number of sets of tomographic image data, and have the same number of pixels within the images. Additionally, the imaging conditions for the first scan and the second scan (imaging positions, rotation speed of the rotator 13, and the like) are the same. In other words, the first volume data and second volume data are on the same coordinate values system. Additionally, in the present embodiment, the axial image AI based on the first volume data, and the axial image AI′ based on the second volume data, are images depicting cross-sections in the same position in the rostrocaudal direction.

The second setter 440 is used to set a new insertion route in regard to the image based on second volume data, if it is determined that misalignment has occurred. The new insertion route is obtained by correcting the planned route (insertion route I) in response to the misalignment.

As a specific example of the second setter 440, there follows a description of a case in which the tip position h of the puncture needle PN is misaligned from the preset insertion route I (see FIG. 7C). FIG. 7C and FIG. 7D depict the axial image AI′ based on the second volume data. In FIG. 7C and FIG. 7D, it is depicted an example in which the tip position h has become misaligned from the insertion route I during puncturing, after inserting the puncture needle PN from the designated insertion position P.

In this case, the second setter 440 sets a new insertion route I′ in the form of a line segment connecting the coordinate values of the tip position h of the puncture needle PN and the coordinate values of one end of the insertion route I (the lesion site position S) (see FIG. 7D). The insertion route I′ should ideally be the shortest route between the tip position h of the puncture needle PN and one end of the insertion route I.

The operator may draw the line segment connecting tip position h of the puncture needle PN and one end of the insertion route I on the axial image AI′ based on the second volume data directly, using an input device, and the like. In this case, the second setter 440 sets the relevant drawn line segment as the new insertion route I′. Alternatively, similarly to the first setter 420, the second setter 440 may set the new insertion route I′ in regard to a coronal image, sagittal image, oblique image and pseudo three-dimensional image, based on the second volume data.

Since the insertion route I is configured on the image based on volume data, the insertion route I possesses three-dimensional coordinate values. As a result, the image on which the insertion route I is set may be different from the image on which the new insertion route I′ is set. For example, the first setter 420 sets the insertion route I on the axial image AI. The second setter 440 then may set the new insertion route I′ on a coronal image.

Furthermore, if the misalignment is small, it may be that there is no impact on the puncturing, and that a new insertion route I′ does not need to be set. In this case, the second setter 440 may set a new insertion route I′ only when the misalignment detected by the determinator 430 is above a threshold value. The threshold value is a value set based on the distance between the insertion route I and the tip position h of the puncture needle PN. Alternatively, the threshold value may be set as an arbitrary value for each application of CT fluoroscopy, using an input device, and the like.

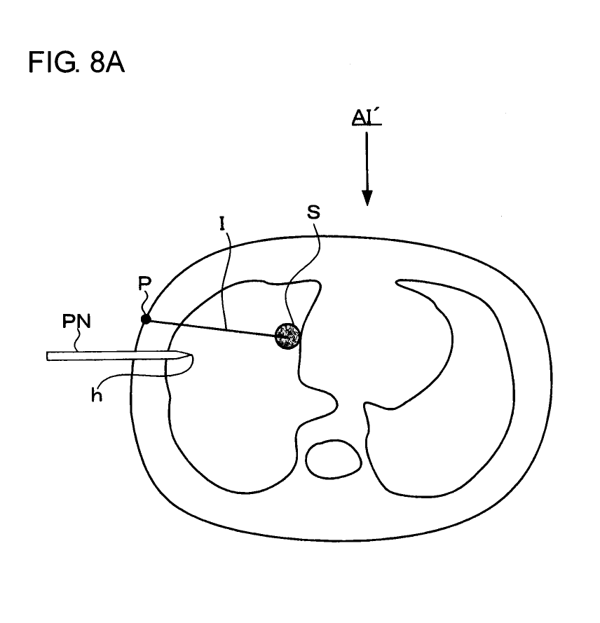

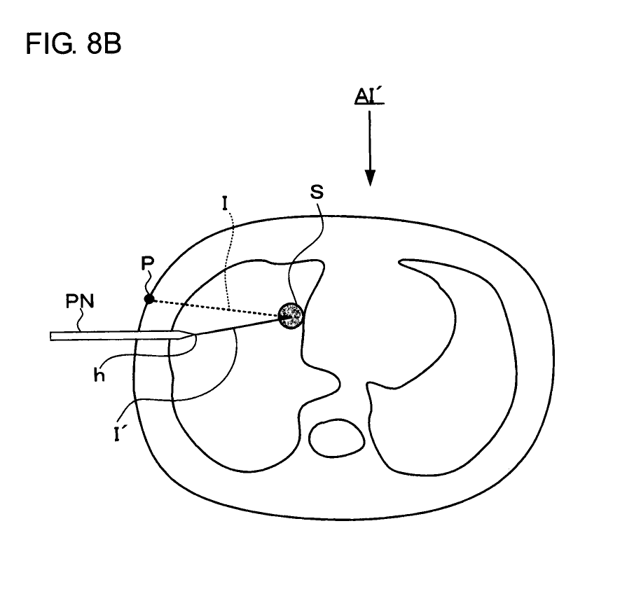

Furthermore, as depicted in FIG. 8A and FIG. 8B, even in cases where the puncture needle PN is significantly misaligned from the insertion position P, the same procedure as that outlined above may be used to set a new insertion route I′. FIG. 8A and FIG. 8B depict the axial image AI′ based on the second volume data.

The display controller 450 implements various controls relating to the image display. For example, the display controller 450 controls the display 470 to display the pseudo three-dimensional image or the MPR image (axial image, sagittal image, coronal image or oblique image) created by the rendering processor 410c.

Furthermore, in the present embodiment, the display controller 450 causes the display 470 to display the image based on volume data, as well as to display the set new insertion route I′ on the image based on volume data.

The following is a description of a case in which the axial image AI′ based on the second volume data is displayed on the display 470 as a specific example of the display controller 450. In this case, the display controller 450 causes the display of the new insertion route I′ set by the second setter 440 in the axial image AI′ (see FIG. 7D). As the display format of the new insertion route I′, the display controller 450 can replace the pixels (pixel value) of the axial image AI′ with the pixels (pixel value) of the new insertion route I′. Alternatively, the display controller 450 may cause the new insertion route I′ to be superimposed on the axial image AI′. Furthermore, the display controller 450 may cause the display of both the original insertion route I and the new insertion route I′ on the axial image AI′ (see FIG. 7D). Alternatively, the display controller 450 may cause the display of only the new insertion route I′ on the axial image AI′.

In addition, the display controller 450 may cause the display of the original insertion route I and the new insertion route I′ in different display formats. For example, the display controller 450 may cause the display of the original insertion route I and the new insertion route I′ in different colors. The display controller 450 may cause the display of the original insertion route I as a flashing display, and the new insertion route I′ as a lit display. The display controller 450 may cause the display of the original insertion route I as a broken line, and the new insertion route I′ as a solid line (see FIG. 7D).

Furthermore, the display controller 450 may cause information indicating a misalignment (for example, the extent of misalignment in terms of the distance or the angle between the tip position h of the puncture needle PN and the insertion route I) to be displayed in numbers or the like in a specified position on the display 470 screen (including cases in which this information is displayed superimposed on the axial image AI′).

The storage 460 is configured to include a semiconductor storing device, such as RAM, ROM, and the like. The storage 460 stores not only the insertion route I position, but also the detection data, projection data, or alternatively the CT image data subsequent to reconstruction processing.

The display 470 is configured to include an arbitrary display device such as an LCD, CRT display device, or the like. The display 47, for example, displays the MPR image obtained by rendering volume data.

The scan controller 480 controls various operations relating to X-ray scanning. The scan controller 480, for example, controls the high voltage generator 140 so as to apply a high voltage to the X-ray generator 110. The scan controller 480 controls the gantry driver 150 so as to rotatably drive the rotator 130. The scan controller 480 controls the collimator driver 170 so as to operate the X-ray collimator 160. The scan controller 480 controls the couch driver 320 so as to transfer the couch 310.

The controller 490 controls the movement of the gantry apparatus 100, couch apparatus 300 and console device 400, thereby controlling the X-ray CT apparatus 1 as a whole. For example, the controller 490 controls the scan controller 480, thereby causing the gantry apparatus 100 to implement the preparatory scan and the main scan, and to acquire detection data. Furthermore, the controller 490 controls the processor 410, thereby causing the processor 410 to implement various types of processing on the detection data (pre-processing, reconstruction processing, and the like). Alternatively, the controller 490 controls the display controller 450, so that the display controller 450 causes the display 470 to display the image on based on the CT image data stored in the storage 460.

### <Operation>

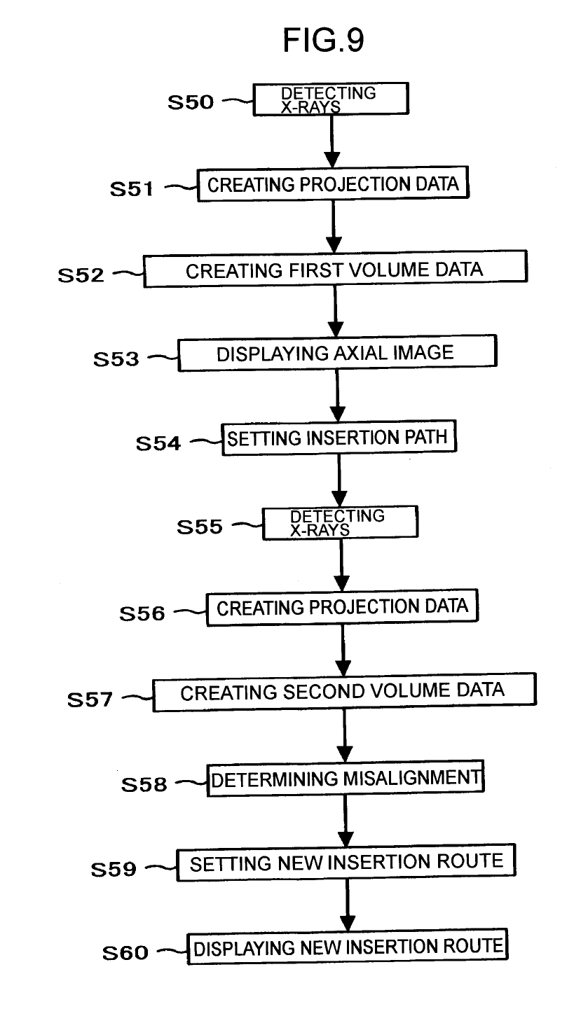

Next, there follows a description of the operation of the X-ray CT apparatus 1 in the present embodiment, in reference to FIG. 9. The explanation given here is of a case in which biopsy is carried out using CT fluoroscopy, once the puncture needle insertion route (planned route) has been set.

Prior to beginning the biopsy, the X-ray CT apparatus 1 first implements an X-ray scan (first scan) on the subject E, and creates volume data (first volume data).

Specifically, the X-ray generator 110 irradiates the subject E with X-rays. The X-ray detector 120 detects the X-rays passing through the subject E, and obtains detection data (S50). The detection data detected by the X-ray detector 120 is acquired by the data acquisition system 180, before being transmitted to the processor 410 (pre-processor 410a).

The pre-processor 410a implements pre-processing, such as logarithmic conversion, offset correction, sensitivity correction, and beam hardening correction, on the detection data obtained in S50, and creates projection data (S51). The projection data created is transmitted to the reconstruction processor 410b based on the control of the controller 490.

The reconstruction processor 410b creates multiple sets of tomographic image data based on the projection data created in S51. Furthermore, the reconstruction processor 410b creates first volume data by interpolation processing of the multiple sets of tomographic image data (S52). The rendering processor 410c creates an axial image AI by rendering the first volume data created in S52. The display controller 450 causes the display 470 to display the created axial image AI (S53).

The operator uses an input device, and the like, to specify a lesion site position S and an insertion position P of the puncture needle PN on the axial image AI, in reference to the axial image AI displayed on the display 470. The first setter 420 sets a line segment connecting the specified positions as an insertion route I (S54. See FIG. 7B). The display controller 450 causes the display of the set insertion route I (planned route) on the axial image AI. The first setter 420 transmits an image of the insertion route I and coordinate values of the insertion route I to the storage 460. The storage 460 stores the relevant image and relevant coordinate values.

Subsequently, the operator begins the biopsy on the subject E, while referring to the axial image AI on which the insertion route I is displayed.

When the biopsy has progressed to a certain extent (once the puncture needle has been inserted into the subject E), the X-ray CT apparatus 1 implements an X-ray scan (second scan) on the subject E in order to confirm the state of the puncture (whether or not the puncture needle PN is following the planned route, and the like), and creates volume data (second volume data).

In other words, similarly to the first scan, the X-ray generator 110 irradiates the subject E with X-rays. The X-ray detector 120 detects the X-rays passing through the subject E, and obtains detection data (S55). As stated above, the first scan and the second scan are implemented under the same imaging conditions.

The pre-processor 410a implements pre-processing on the detection data obtained in S55, and creates projection data (S56). The reconstruction processor 410b implements interpolation processing on the multiple sets of tomographic image data created based on the projection data created in S56, and creates the second volume data (S57). The rendering processor 410c renders the second volume data created in S57 to create an axial image AI′. The axial image AI′ represents a cross-section at the same position in the rostrocaudal direction as the axial image AI displayed in S53.

At this point, the determinator 430 determines whether any misalignment has occurred between a tip position h of the puncture needle PN and the insertion route I within the axial image AI′ (S58).

If it is determined in S58 that misalignment has occurred, the second setter 440 sets a new insertion route I′ in regard to the axial image AI′ (S59). On the other hand, if it is determined that there is no misalignment, then since the puncture needle is progressing according to plan, the processes of the X-ray CT apparatus 1 outlined from S59 onwards are not required.

The display controller 450 causes the display 470 to display the axial image AI′, as well as to display the new insertion route I′ set in S59 on the axial image AI′ (S60).

The processor 410, setter 420, determinator 430, second setter 440, display controller 450, scan controller 480 and controller 490 may be configured from processing apparatus, not depicted in the diagrams, such as a CPU, GPU and ASIC, or storage apparatus, not depicted, such as a ROM, RAM a HDD. The storing device stores processing programs that enable the processor 410 to implement its functions. Furthermore, the storing device stores setter processing programs that enable the first setter 420 and the second setter 440 to implement their functions. Additionally, the storing device stores determinator processing programs that allow the determinator 430 to implement its functions. Furthermore, the storing device stores display control programs that enable the display controller 450 to implement its functions. In addition to this, the storing device stores scan control programs that enable the scan controller 480 to implement its functions. Additionally, the storing device stores control programs that enable the controller 490 to implement its functions. The processing apparatus of the CPU and the like implement functions of various devices by implementing each program stored in the storage apparatus.

In the present embodiment, the functions of the first setter 420 and the second setter 440 have been described separately. Alternatively, a single setter may be provided to implement each function (the operations of the first setter 420 and the second setter 440).

Furthermore, the configuration and operation of a single X-ray CT apparatus 1 has been described to this point. Alternatively, the configuration of the present embodiment may be realized using an X-ray CT system including an X-ray CT apparatus 1.

For example, an insertion route I is set in an image based on pre-created volume data in the X-ray CT apparatus 1, and an insertion route I image and an insertion route I position are stored. Next, a CT fluoroscopy biopsy is performed using another X-ray CT apparatus. In this case, the other X-ray CT apparatus reads the stored insertion route I from the X-ray CT apparatus 1, and determines whether there is any misalignment between the puncture needle PN and the insertion route I in the image based on a new volume data (second volume data) obtained in CT fluoroscopy. If there is a misalignment, the other X-ray CT apparatus sets a new insertion route I′ on the image based on the second volume data. The other X-ray CT apparatus then causes the display to display the image based on the second volume data, as well as the new insertion route I′ on the image.

Alternatively, in the X-ray CT apparatus 1, an image based on the first volume data is created. A computer that is separate to the X-ray CT apparatus 1 sets the insertion route I in the image based on the first volume data, and stores the insertion route I image and insertion route I position. Next, if the X-ray CT apparatus 1 (or another X-ray CT apparatus) is to be used to implement CT fluoroscopy, the X-ray CT apparatus 1 reads the stored insertion route I from the computer, and determines whether there is any misalignment between the puncture needle PN and the insertion route I in the image based on the second volume data obtained in CT fluoroscopy. If there is a misalignment, the X-ray CT apparatus 1 sets a new insertion route I′ on the image based on the second volume data. The X-ray CT apparatus 1 then causes the display to display the image based on the second volume data, as well as the new insertion route I′ on the image.

### <Operation and Effect>

The following is a description of the operation and effect of the present embodiment.

The X-ray CT apparatus 1 in the present embodiment creates first volume data and second volume data based on the results of scanning the subject E with X-rays. The X-ray CT apparatus 1 comprises a first setter 420, a determinator 430, a second setter 440 and a display controller 450. The first setter 420 is used to set an insertion route I of a puncture needle PN in regard to the subject E, in the image based on a pre-created first volume data. The determinator 430 determines whether there is any misalignment between the puncture needle PN and the insertion route I in the image based on a second volume data created based on result of the scan taken when the puncture needle PN is inserted into the subject E. The second setter 440 is used to set a new insertion route I′ on the image based on the second volume data, in cases where it is determined that a misalignment has occurred. The display controller 450 causes the display 470 to display the image based on the second volume data, as well as causing the set new insertion route I′ to be displayed in the image based on the second volume data.

In addition, the configuration of the present embodiment may be realized as an X-ray CT system. The X-ray CT system includes an X-ray CT apparatus 1, which creates volume data based on the results of scanning the subject E with X-rays. The X-ray CT system comprises a first setter 420, a determinator 430, a second setter 440 and a display controller 450. The first setter 420 is used to set an insertion route I of a puncture needle PN in regard to a subject E, in the image based on a pre-created first volume data. The determinator 430 determines whether there is any misalignment between the puncture needle PN and the insertion route I in the image based on a second volume data created based on the result of the scan taken when the puncture needle PN is inserted into the subject E. The second setter 440 is used to set a new insertion route I′ on the image based on the second volume data, in cases where it is determined that a misalignment has occurred. The display controller 450 causes the display 470 to display the image based on the second volume data, as well as causing the set new insertion route I′ to be displayed in the image based on the second volume data.

In this way, if there is any misalignment between the puncture needle PN and the insertion route I, the second setter 440 sets a new insertion route I′. The display controller 450 causes the display of the new insertion route I′ on an image based on the volume data. The operator can easily ascertain how to insert the puncture needle in regard to the site to which biopsy is performed referring to this image. In other words, using the X-ray CT apparatus (X-ray CT system) in the present embodiment, it is possible to display an image that reflects the misalignment between the planned route and the puncture needle.

Additionally, the display controller 450 in the X-ray CT apparatus 1 in the present embodiment causes the display of insertion route I set by the first setter 420 on the image based on the second volume data.

As described above, displaying the new insertion route I′ on the image based on the second volume data together with the pre-set insertion route I in this way allows the operator to easily ascertain any misalignment between the pre-set insertion route I and the new insertion route I′.

In addition, the display controller 450 in the X-ray CT apparatus 1 in the present embodiment causes the display 470 to display information indicating the misalignment.

As described above, displaying information indicating the misalignment on the display 470 in this way allows the operator to specifically ascertain the misalignment as information expressed in numbers, and the like.

Additionally, the display controller 450 in the X-ray CT apparatus 1 in the present embodiment causes the display of the insertion route I and the new insertion route I′ in different formats.

Displaying the insertion route I and the new insertion route I′ in different formats in this way allows each route to be easily distinguished. As a result, the operator can easily determine a path along which the puncture needle PN is to be inserted.

### Fourth Embodiment

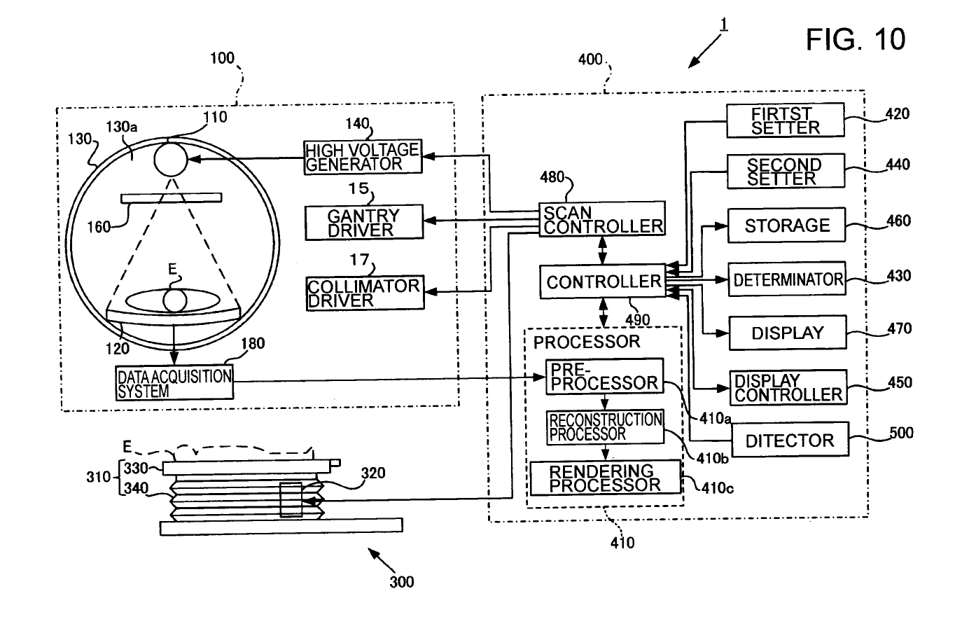

The following is a description of the X-ray CT apparatus 1 in a fourth embodiment, in reference to FIG. 10 through FIG. 13. In a case of implementing a biopsy on a subject E, for example, it is desirable to avoid blood vessels, and the like, when inserting a puncture needle. The present embodiment describes a configuration by which a puncture needle insertion route and new insertion route are set while avoiding blood vessels, and the like. Details of the configuration that are the same as the third embodiment have been omitted from this description.

A console device 400 in the present embodiment is configured to include a processor 410, a first setter 420, a determinator 430, a second setter 440, a display controller 450, a storage 460, a display 470, a scan controller 480, a controller 490, and a detector 500.

The detector 500 detects a specified target site from volume data. The “specified target site” is an identified site, within the subject E, included in volume data of blood vessels and the like. The target site is a site that should be avoided from being punctured with the puncture needle (in other words, it is desirable for the insertion route to be set so as to avoid the target site). The detected target site may be stored in the storage 46, and the like, if set in advance, or may be set as an arbitrary site using an input device, and the like, each time a biopsy is carried out. Furthermore, the target site may be a site, or it may be a point that is the smallest unit within an area (for example, a voxel (pixel) with the highest CT value within the volume data).

The following is a specific example of the detector 500, with a configuration in which the target site is detected from an MPR image created based on a first volume data. The detector 500 compares the CT values of each pixel in the MPR image with the threshold value for the target site being detected. Subsequently, the detector 500 detects pixels (pixel coordinate values) with CT values above the threshold value (or below the threshold value) as the target site (target site coordinate values). The threshold value is a value determined corresponding to the target site (for example, CT values of blood vessels), and is a value that is used to determine whether the target site is included within the pixel or not. The threshold value may have a specified width. If the threshold value has a width, the detector 500 detects pixels with CT values included within the threshold value as the target site.

The detector 500 may detect the target site directly from volume data. In this case, the detector 500 compares the CT values of each voxel that makes up the volume data with the threshold value of the target site to be detected. Subsequently, the detector 500 detects voxels (voxel coordinate values) with CT values above the threshold value (or below the threshold value) as the target site (target site coordinate values).

The insertion route is s to avoid the target site detected from the first volume data using the first setter 420 in the present embodiment.

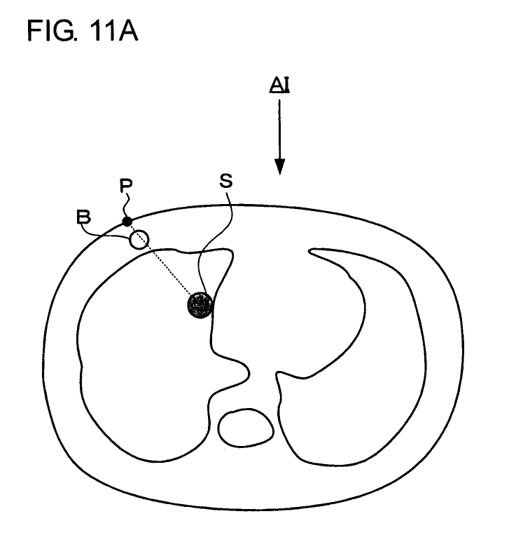

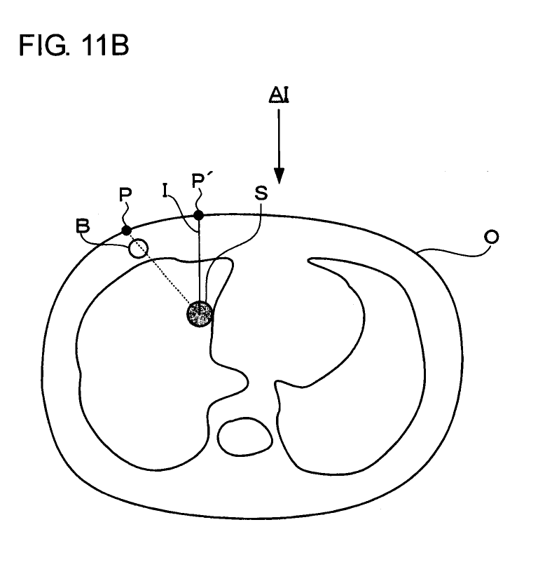

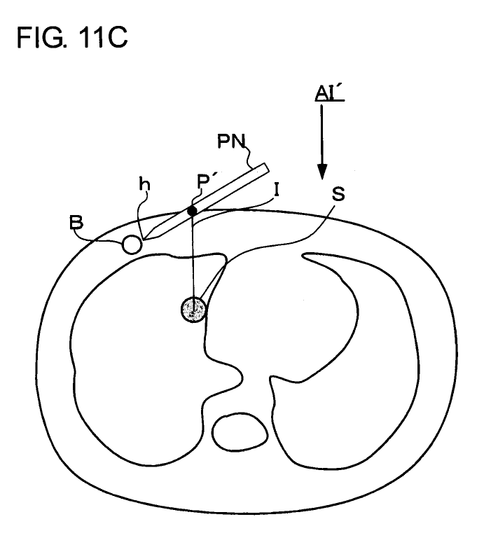

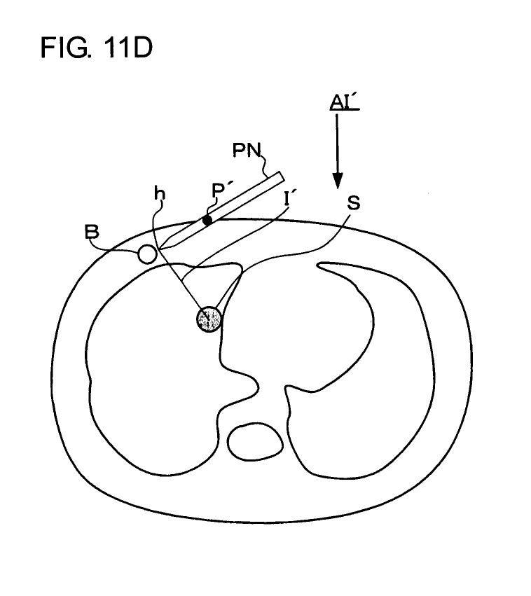

FIG. 11A depicts an axial image AI based on the first volume data. Here, if the insertion route (see broken line in FIG. 11A) is set taking the shortest distance between an insertion position P, where the puncture needle is to be inserted, and a lesion site position S, there exists blood vessels B on the insertion route (see FIG. 11A). As a result, if the puncture is carried out along the insertion route, the blood vessels B will be punctured.

Therefore, the first setter 420 implements image analysis processing, such as edge detection, to calculate the lesion site position S and a contour O of the body surface within the axial image AI. The first setter 420 then specifies a point P′ at which the position S comes closest to the contour O (in other words, where the distance between position S and point P′ is the shortest distance between position S and contour O). Here, the first setter 420 determines whether or not there are blood vessels B present on the line segment that connects the position S and the point P′. In other words, the first setter 420 determines whether the coordinate values of any blood vessels B detected by the detector 500 are included within the coordinate values of the line segment. If it is determined that no blood vessels B are present on the line segment that connects the position S and the point P′ (the coordinate values of blood vessels B are not included within the coordinate values of the line segment), the first setter 420 sets the insertion route I along the line segment (see FIG. 11B). On the other hand, if it is determined that blood vessels B are present on the line segment that connects the position S and the point P′ (the coordinate values of blood vessels B are included within the coordinate values of the line segment), the first setter 420 specifies a new point on the contour O, and then determines once again whether or not there are blood vessels B present on the line segment that connects the position S and the newly specified point.

The insertion route I can be set in any way providing the route avoids blood vessels B, and does not need to be the shortest distance between the position S and the contour O. In other words, the coordinate values of the insertion route I may be anything other than the coordinate values of the blood vessels B.

Furthermore, if the operator uses an input device, and the like, to draw the line segment depicting the insertion route I directly onto the axial image AI, it is possible that the insertion route I may overlap the detected target site (blood vessels B, and the like.) In this case, the X-ray CT apparatus 1 may issue a warning to indicate that the set insertion route I is not desirable. For example, the display controller 450 may cause the display 470 to display a warning such as “Need to change insertion route”. Alternatively, the controller 490 can operate a warning procedure (not depicted), and issue a sound warning.

In the present embodiment, the second setter 440 is used to set a new insertion route that avoids the target site detected from the first volume data or the second volume data. FIG. 11C and FIG. 11D depict an axial image AI′ based on the second volume data. FIG. 11C and FIG. 11D depict an example wherein the tip position h of the puncture needle PN has become misaligned from the insertion route I during puncturing, after inserting the puncture needle PN from the designated insertion position P.

As shown in the example in FIG. 11C, for example, in a case that the puncture needle PN is misaligned from the insertion route I, it may puncture the blood vessels B by further performing puncturing. For this reason, the second setter 440 sets a new insertion route I′, which is set avoiding the blood vessels B. Specifically, the second setter 440 identifies the shortest route between the tip position h of the puncture needle PN and the lesion site position S, and determines whether or not there are blood vessels B present on this shortest route. If it is determined that there are no blood vessels B present, the second setter 440 sets this identified shortest route as the new insertion route I′ (see FIG. 11D).

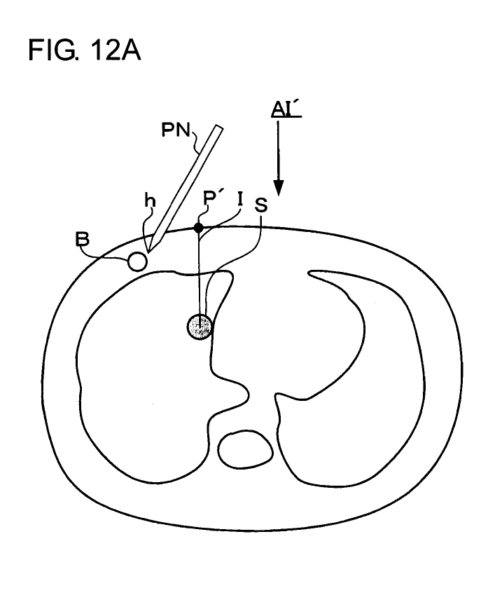

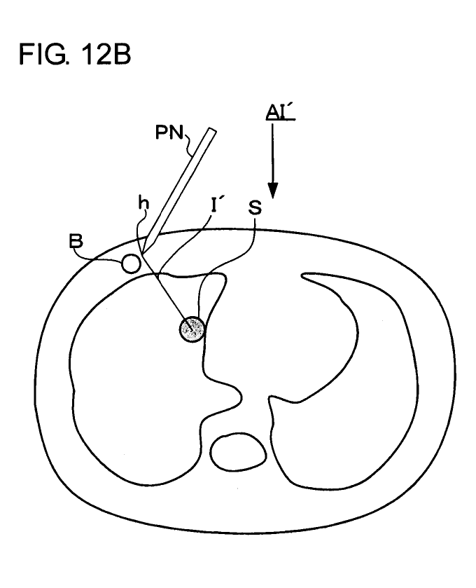

Furthermore, as depicted in FIG. 12A and FIG. 12B, the second setter 440 can set the new insertion route I′ using the same processes as those described above even if the puncture needle PN is punctured at a position significantly misaligned from the insertion position P. FIG. 12A and FIG. 12B depict an axial image AI′ based on the second volume data.

The detector 500 may detect a target site each time an X-ray scan is performed. It is possible, for example, that the position of the target site, or the like, may change depending on the timing of the first volume data acquisition and the second volume data acquisition, depending on the effect of breathing or the pulse.

In such cases, the detector 500 detects the specified target site once again based on the second volume data obtained at a different timing to the first volume data. The second setter 440 then identifies the line segment connecting the tip position h of the puncture needle PN and the lesion site position S to avoid the target site detected in the second volume data, and sets the new insertion route I′ in line with the line segment. In this way, the second setter 440 sets the new insertion route I′ that avoids the target site detected from the image based on the second volume data. As a result, the X-ray CT apparatus 1 is able to set the new insertion route I′ with the minimum impact from changes of the position of blood vessels B, and the like.

### <Operation>

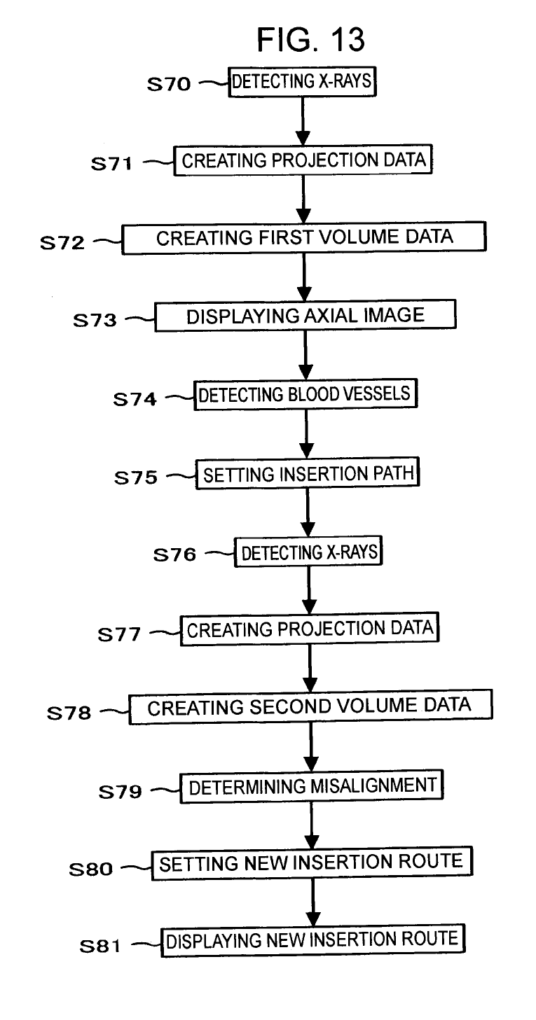

The following is a description of the operation of the X-ray CT apparatus 1 in the present embodiment, in reference to FIG. 13. Here, the description is of the operation wherein a biopsy is implemented using CT fluoroscopy, after setting the puncture needle insertion route (planned route).

Prior to beginning the biopsy, the X-ray CT apparatus 1 first implements an X-ray scan (first scan) on the subject E, and creates volume data (first volume data).

Specifically, the X-ray generator 110 irradiates the subject E with X-rays. The X-ray detector 120 detects the X-rays passing through the subject E, and obtains detection data (S70). The pre-processor 410a implements pre-processing, such as logarithmic conversion, offset correction, sensitivity correction, and beam hardening correction, on the detection data obtained in S70, and creates projection data (S71). The reconstruction processor 410b creates multiple sets of tomographic image data based on the projection data created in S71. Furthermore, the reconstruction processor 410b creates the first volume data by interpolation processing of the multiple sets of tomographic image data (S72). The rendering processor 410c creates an axial image AI by rendering the first volume data created in S72. The display controller 450 causes the display 470 to display of the created axial image AI (S73).

At this point, the detector 500 compares CT values of each pixel in the axial image AI with a threshold value for blood vessels B, to detect the blood vessels B within the axial image AI (S74).

The first setter 420 determines a lesion site position S and a contour O of the body surface within the axial image AI by performing edge detection, and the like. The first setter 420 then identifies a point P′ at which the position S comes closest to the contour O. The first setter 420 determines whether or not there are blood vessels B present on a line segment that connects the position S and the point P′. If it is determined that no blood vessels B are present on the line segment that connects the position S and the point P′, the first setter 420 sets the insertion route I along the line segment. In other words, the first setter 420 sets the insertion route I so as to avoid blood vessels B detected in S74 (S75). The display controller 450 causes the display of the set insertion route I on the axial image AI. The first setter 420 transmits the insertion route I image and the insertion route I coordinate values to the storage 460. The storage 460 stores the image and the coordinate values.

Subsequently, the operator begins to biopsy with respect to the subject E, referring to the axial image AI depicting the insertion route I.

When the biopsy has progressed to a certain extent (once the puncture needle PN has been inserted into the subject E), the X-ray CT apparatus 1 implements a further X-ray scan (second scan) on the subject E in order to confirm the state of the puncture (whether or not the puncture needle PN is following the planned route, and the like), and creates volume data (second volume data).

In other words, similarly to the first scan, the X-ray generator 110 irradiates the subject E with X-rays. The X-ray detector 120 detects the X-rays passing through the subject E, and obtains detection data (S76). As stated above, the first scan and the second scan are implemented under the same imaging conditions.

The pre-processor 410a implements pre-processing on the detection data obtained in S76, and creates projection data (S77). The reconstruction processor 410b implements interpolation processing on multiple sets of tomographic image data created based on the projection data created in S77, and creates the second volume data (S78). The rendering processor 410c renders the second volume data created in S78 to create an axial image AI′. This axial image AI′ depicts a cross-section of the same position in the rostrocaudal direction as the axial image AI depicted in S73.

At this point, the determinator 430 determines whether there is any misalignment, in the axial image AI′, between the tip position h of the puncture needle PN and the insertion route I (S79).

If it is determined in S79 that misalignment has occurred, the second setter 440 sets a new insertion route I′, avoiding blood vessels B detected in S74, on the axial image AI′ (S80). On the other hand, if it is determined that no misalignment has occurred, the X-ray CT apparatus 1 does not implement any of the processes subsequent to S80, since the puncturing is proceeding as planned.

The display controller 450 causes the display 470 to display the axial image AI′, as well as causing the new insertion route I′ set in S80 to be displayed on the axial image AI′ (S81).

### <Operation and Effect>

The following is a description of the operation and effect of the present embodiment.

The X-ray CT apparatus 1 in the present embodiment includes a detector 500. The detector 500 detects a specified target site (for example, blood vessels) from volume data. The first setter 420 sets an insertion route I to avoid the target site detected from a first volume data. The second setter 440 sets a new insertion route I′ to avoid the target site detected from the first volume data or the second volume data.

In this way, the first setter 420 sets the insertion route I to avoid blood vessels, and the like (the target site should to be avoided to be punctured) detected by the detector 500. Furthermore, if any misalignment occurs between the puncture needle PN and the insertion route I, the second setter 440 sets a new insertion route I′ that avoids blood vessels, and the like. In other words, the X-ray CT apparatus (X-ray CT system) in the present embodiment makes possible the display of an image that reflects misalignment between the planned route and the puncture needle. Furthermore, this image is an image set by avoiding blood vessels, and the like. Referring to this image while implementing puncturing allows the operator to reduce the possibility of puncturing blood vessels, and the like. In other words, the X-ray CT apparatus (X-ray CT system) in the present embodiment makes possible the provision of an image that can be referred to when puncturing in such a way as to avoid blood vessels, and the like.

<Common Effects within the Third Embodiment and Fourth Embodiment>

Of the third embodiment and fourth embodiment described above, in the X-ray CT apparatus in at least one of the embodiments, if any misalignment occurs between a puncture needle and an insertion route, the second setter sets a new insertion route. The display controller causes the display of the new insertion route in an image based on volume data. In other words, the X-ray CT apparatus in these embodiments makes possible the display of an image reflecting misalignment between a planned route and the puncture needle.

While certain embodiments have been described are set forth; however, the embodiments described above were presented as examples and are not intended to limit the scope of the invention. These new embodiments may be carried out in various other configurations, and various abbreviations, replacements, and changes may be made in a range not departing from the summary of the invention. These embodiments and deformations thereof are included in the range and summary of the invention and included in the invention described in the range of patent claims as well as the range of the equivalent thereof.

### EXPLANATION OF SYMBOLS

1 X-ray CT apparatus
10 Gantry apparatus
11 X-ray generator
12 X-ray detector
13 Rotator
13a Opening
14 High voltage generator
15 Gantry driver
16 X-ray collimator
17 Collimator driver
18 Data acquisition system
30 Couch apparatus
32 Couch driver
33 Couch top
34 Base
40 Console device
41 Processor
41a Pre-processor
41b Reconstruction processor
41c Rendering processor
411c First image processor
412c Second image processor
42 Setter
43 Storage
44 Display controller
45 Display
46 Scan controller
47 Controller
E Subject

1. An X-ray CT apparatus configured to create first volume data and second volume data based on the results of scanning a subject with X-rays at different timings, comprising:
a setter configured to set a specified setup image in regard to an image based on the first volume data, and
a storage configured to store the setup image and a setup position thereof, and
a display controller configured to cause a display to display an image based on the second volume data, as well as causing the setup image to be displayed in the position corresponding to the setup position in the image based on the second volume data.

2. The X-ray CT apparatus according to claim 1, comprising:
a first image processor configured to create a pseudo three-dimensional image depicting the three-dimensional structure of the subject in 2 dimensions based on volume data, wherein
the setter is configured to set the setup image in regard to a pseudo three-dimensional image based on the first volume data, and
the display controller is configured to cause the display to display a pseudo three-dimensional image based on the second volume data, as well as causing the setup image to be displayed in the position corresponding to the setup position in the pseudo three-dimensional image based on the second volume data.

3. The X-ray CT apparatus according to claim 1, comprising:
a second image processor configured to create an MPR image depicting a cross-section of the subject based on volume data, wherein
the setter is configured to set the setup image in regard to a MPR image based on the first volume data, and
the display controller is configured to cause the display to display a MPR image based on the second volume data, as well as causing the setup image to be displayed in the position corresponding to the setup position in the MPR image based on the second volume data.

4. The X-ray CT apparatus according to claim 1, comprising:
a first image processor configured to create a pseudo three-dimensional image depicting the three-dimensional structure of the subject in two dimensions, based on volume data, and
a second image processor configured to create an MPR image depicting a cross-section of the subject based on volume data, wherein
the setter is configured to set the setup image in regard to a MPR image based on the first volume data, and
the display controller is configured to cause the display to display a pseudo three-dimensional image based on the second volume data, as well as causing the setup image to be displayed in the position corresponding to the setup position in the pseudo three-dimensional image based on the second volume data.

5. The X-ray CT apparatus according to claim 3, wherein the second image processor is configured to create at least one out of an axial image, a sagittal image, a coronal image, and an oblique image of the subject as the MPR image.

6. The X-ray CT apparatus according to claim 1, wherein the setup image is an image depicting an insertion route of a puncture needle in regard to the subject.

7. An X-ray CT system comprising an X-ray CT apparatus, configured to produce volume data based on the results of scanning a subject with X-rays, comprising:
a setter configured to set a specified setup image in regard to an image based on a pre-created first volume data, and
a storage configured to store the setup image and a setup position thereof, and
a display controller configured to cause the display to display an image based on a newly created second volume data, as well as causing the display of the setup image in the position corresponding to the setup position in the image based on the second volume data.

8. An X-ray CT apparatus configured to create volume data based on the results of scanning a subject with X-rays, comprising:
a first setter configure to set an insertion route of a puncture needle in regard to the subject in an image based on a pre-created first volume data, and
a determinator configured to determine the existence or otherwise of misalignment between the puncture needle and the insertion route in a image based on a second volume data created based on the results of a scan implemented while the puncture needle is inserted into the subject, and
a second setter configured to set a new insertion route in regard to the image based on the second volume data in cases where the misalignment is determined to exist, and
a display controller configured to cause the display of the image based on the second volume data on a display, as well as causing the display of the newly set insertion route in the image based on the second volume data.

9. The X-ray CT apparatus according to claim 8, comprising:
a detector configured to detect a specified target site from volume data, wherein
the insertion route is configured to set by the first setter to avoid the target site detected from the first volume data, and
the new insertion route is configured to set by the second setter to avoid the target site detected from the first volume data or the second volume data.

10. The X-ray CT apparatus according to claim 8, wherein the display controller is configured to cause the display of the insertion route on the image based on the second volume data.

11. The X-ray CT apparatus according to claim 8, wherein the display controller is configured to cause the display to display information depicting the misalignment.

12. The X-ray CT apparatus according to claim 8, wherein the display controller is configured to cause the display of the insertion route and the new insertion route in different display formats.

13. The X-ray CT apparatus according to claim 8, wherein the display controller configured to cause the display of at least one of an axial image, a sagittal image, a coronal image and an oblique image of the subject as the image based on the first volume data or the image based on the second volume data.

14. An X-ray CT system comprising an X-ray CT apparatus, configured to create volume data based on the results of scanning a subject with X-rays, comprising:
a first setter configure to set an insertion route of a puncture needle in regard to the subject in an image based on a pre-created first volume data, and
a determinator configured to determine the existence or otherwise of misalignment between the puncture needle and the insertion route in a image based on the second volume data created based on the results of a scan implemented while the puncture needle is inserted into the subject, and
a second setter configured to set a new insertion route in regard to the image based on the second volume data in cases where the misalignment is determined to exist, and
a display controller configured to cause the display of the image based on the second volume data on a display, as well as causing the display of the newly set insertion route in the image based on the second volume data.
